(19) United States
(12) Patent Application Publication
(10) Pub. No.: US 2009/0318329 A2
(43) Pub. Date: Dec. 24, 2009

(54) METHODS FOR ASSESSING SUITABILITY OF CANCER PATIENTS FOR TREATMENT WITH HISTONE DEACETYLASE INHIBITORS

(75) Inventor: Qiang Yu, Singapore (SG)

Correspondence Address: CLARK & ELBING LLP, 101 FEDERAL STREET, BOSTON, MA, 02110, UNITED STATES, 617-428-0200, 617-428-7045, patentadministrator@clarkelbing.com

(73) Assignee: Agency for Science, Technology and Research, Singapore (SG)

(21) Appl. No.: 11/884,105

(22) Filed: Jun. 24, 2008

(30) Foreign Application Priority Data
Mar. 10, 2005 (SG) 200501603-5

(51) Int. Cl. A61K 38/02; G01N 33/68; C12Q 1/02; G01N 33/53; A61P 35/00; C12Q 1/68; A61K 31/164

(52) U.S. Cl. 514/2; 436/86; 514/619; 514/616; 435/29; 435/6; 436/501

(57) ABSTRACT

This invention is in the field of cancer therapy and provides the use of E2F1 activity for assessing suitability of a cancer patient for treatment with histone deacetylase inhibitors (HDACIs).

### FIELD OF THE INVENTION

This invention is in the field of cancer therapy and provides the use of E2F1 activity for assessing suitability of a cancer patient for treatment with histone deacetylase inhibitors (HDACIs).

All documents cited in this text (“herein cited documents”) and all documents cited or referenced in herein cited documents are incorporated by reference in their entirety for all purposes.

There is no admission that any of the various documents etc. cited in this text are prior art as to the present invention.

### BACKGROUND

Histone deacetylase inhibitors (HDACIs) have emerged recently as promising chemotherapeutic agents and can induce a range of antitumor activities, including induction of cell cycle arrest, stimulation of differentiation, and provocation of apoptosis (1-3). The efficacy of these agents, particularly Trichostatin A (TSA) and suberoylanilide hydroxamic acid (SAHA) has been established by in vitro experiments and ongoing clinical trials (4-9). Unlike conventional chemotherapeutic agents that often cause DNA damage in both tumor and normal tissues, HDACIs display strong tumor selectivity and cause less toxicity to the normal tissues (2). However, the mechanism of this tumor selectivity is not understood, though recent studies show that HDACI sensitivity in tumor could be mediated by the activation of the death receptor pathway involving the tumor necrosis factor-related apoptosis-inducing ligand (TRAIL)(10, 11) or preferential induction of oxidative injury in transformed cells (12).

The therapeutic effect of HDACIs might be mediated through modulation of chromatin structure and transcriptional activity via changes in the acetylation status of nucleosomal histones at gene promoters. In addition to chromatin remodeling, HDACIs activity may also be linked with non-histone proteins important for growth and differentiation, such as tumor suppressor p53 (13). However, HDACIs induce histone hyperacetylation in both tumor and normal tissues. Thus, altered gene expression patterns through histone/chromatin modulation might not be the primary mechanism to confer cancer selectivity of HDACIs. Alternatively, the tumor selectivity of HDACIs could be related to the chromatin modifications that are associated with oncogenic transformation, which in turn activates an apoptosis program normally suppressed during oncogenesis, an innate tumor suppressive mechanism coupled to oncogenic signaling (14). As a result, cancer cells harboring oncogenic lesions are more susceptible to the cytotoxic effects of HDAC inhibitors.

One such oncogenic lesion lies in the Rb/E2F1 pathway. The loss of Rb tumor suppressor gene has been reported in many human tumors (15). The Rb tumour suppressor regulates proliferation and survival by modulating the activity of E2F transcription factors. The E2F family of transcription factors plays a critical role in overall cell cycle control. Members of the E2F family of transcription factors control cell proliferation by regulating the expression of genes required for S phase entry and progression (59-60).

Hypophosphorylated Rb binds to and sequesters the transcription factor E2F, resulting in the repression of proliferation-associated genes Inactivation of Rb results in increased E2F1 activity and subsequent transactivation of genes required for cell cycle progression, leading to aberrant cell proliferation (16). While Rb disruption primarily occurs in retinoblastoma, Rb inactivation can be caused in many tumor types by alterations of other components in this regulatory machinery, such as loss of p16(INK4), or overexpression of cyclin D1 and Cdk4. In addition, increased-E2F1 expression has also been observed in several types of human tumors including breast cancer, non-small cell lung cancer and salivary gland tumor (17-19). Therefore, the activation of E2F1 activity through various mechanisms allows tumor cells to evade cell cycle regulation and proliferate uncontrollably. Accordingly, disruption of the normal Rb-E2F function is regarded as one of the most frequent alterations of malignant transformation (20). As a fail-safe mechanism to protect aberrant oncogenic transformation (14), E2F1 is also equipped with a tumor suppressor function by inducing apoptosis. Through this mechanism, cells with mutations in the Rb-E2F pathway will be predisposed to die and to be cleared. Indeed, deregulated E2F activity can trigger apoptosis through regulating the expression of pro-apoptotic genes (21, 22). These include the induction of p19^{ARF} (23, 24) or Chk2 (25) and subsequently activation of p53-dependent apoptotic pathway. E2F1 also induces the expression of p73 (26, 27), Caspases (28) and pro-apoptotic BH3-only proteins of Bcl-2 family (29) and thus induces apoptosis through a p53-independent mechanism. To allow malignant outgrowth, the oncogene-coupled apoptosis function is either disrupted or inactivated. Therefore, therapeutic approaches for fully activating oncogene-induced apoptosis appear to be conceptually feasible to achieve tumor-specific intervention.

### SUMMARY

In this study, we demonstrate that HDACIs promote apoptosis through activation of the oncogenic Rb/E2F1 pathway and that cancer cells with increased E2F1 activity or Rb inactivation are highly susceptible to HDACIs-induced cell death. We show that the proapoptotic Bcl-2 family member Bim is a key mediator of this apoptotic process Our results provide a mechanistic explanation for the tumor selectivity of HDACIs and suggest that HDACIs right preferentially kill tumors with deregulated Rb-E2F1 pathway.

We also investigated the transcriptional response of apoptotic network to HDAC inhibitor SAHA that is affected by E2F1 activity and identified ASK1 as an additional target of E2F1 that participates in HDACI-induced cell death Contrary to an established role of ASK1 in regulating its downstream apoptotic signaling pathways, we show that ASK1 induction contributes to SAHA-induced apoptosis through a positive feedback regulation of E2F1 apoptotic activity.

### GLOSSARY OF TERMS

This section is intended to provide guidance on the interpretation of the words and phrases set forth below (and where appropriate grammatical variants thereof). Further guidance on the interpretation of certain words and phrases as used herein (and where appropriate grammatical variants thereof) may additionally be found in other sections of this specification.

As used herein, the singular form “a,” “an,” and “the” include plural references unless the context clearly dictates otherwise. For example, the term “an agent” includes a plurality of agents, including mixtures thereof and reference to “the nucleic acid sequence” generally includes reference to one or more nucleic acid sequences and equivalents thereof known to those skilled in the art, and so forth.

As used herein, the term “about” as used in relation to a numerical value means, for example, ±50% of the numerical value, preferably ±20%, more preferably ±10%, more preferably still ±5%, and most preferably ±1%. Where necessary, the word “about” may be omitted from the definition of the invention.

The term “antibody” means an immunoglobulin molecule able to bind to a specific epitope on an antigen. Antibodies can be comprised of a polyclonal mixture, or may be monoclonal in nature. Further, antibodies can be entire immunoglobulins derived from natural sources, or from recombinant sources. The antibodies used in the present invention may exist in a variety of forms, including for example as a whole antibody, or as an antibody fragment, or other immunologically active fragment thereof, such as complementarity determining regions Similarly, the antibody may exist as an antibody fragment having functional antigen binding domains, that is, heavy and light chain variable domains. Also, the antibody fragment may exist in a form selected from the group consisting of, but not limited to: Fv, F_{ab}, F(ab)₂, scFv (single chain Fv), dAb (single domain antibody), bi-specific antibodies, diabodies and triabodies.

Ask herein, an “array” includes an intentionally created collection of molecules (e.g. probes) which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports. The term “array” includes, inter alia, those libraries of nucleic acids which can be prepared by spotting nucleic acids of essentially any length (e.g., from 1 to about 1000 nucleotide monomers in length) onto a substrate. As used herein, the term array and microarray may be used interchangeably.

The term a “cancer patient” includes any patient who is need of anti-cancer treatment. The term may include an individual suspected of suffering from cancer, or an individual suspected of being predisposed to cancer, or an individual who may have previously suffered from cancer or an individual who may currently be suffering from cancer.

The term “complementary” refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100% of the nucleotides of the other strand. Alternatively, complementarity exists when an RNA or DNA strand will hybridize under selective hybridization conditions to its complement. Typically, selective hybridization will occur when there is at least about 65% complementarity over a stretch of at least 14 to 25 nucleotides, preferably at least about 75%, and more preferably at least about 90% complementarity.

As used herein, the term “comprising” means “including”. Thus, for example, a composition “comprising” X may consist exclusively of X or may include one or more additional components.

As used herein, the terms “histone deacetylase” and “HDAC” are intended to refer to any one of a family of enzymes that remove acetyl groups from the E-amino groups of lysine residues at the N-terminus of a histone. Unless otherwise indicated by context, the term “histone” is meant to refer to any histone protein, including H1, H2A, H2B, H3, H4, and H5 from any species to be treated. Preferred histone deacetylases include class I and class 11 enzymes. Preferably the HDAC is a mammalian or human HDAC. Human HDACs include HDAC-1, HDAC-2, HDAC-3, HDAC-4, HDAC-5, HDAC-6, HDAC-7, HDAC-8, HDAC-9, HDAC-10, and HDAC-11.

The terms “histone deacetylase inhibitor”, “inhibitor of histone deacetylase” and “HDACIs” are used interchangeably and includes compounds which are capable of interacting with a histone deacetylase and inhibiting its enzymatic activity. “Inhibiting histone deacetylase enzymatic activity” means reducing the ability of a histone deacetylase to remove an acetyl group from a histone.

The person skilled in the art can select suitable compounds on the basis of the known structures (and amino acid sequences) of histone deacetylases, e.g. histone deacetylases 1, 2, 3, 4, 5, 6, 7, 7A, isoform a, 7B, isoform b and 8; see NCBI-Databases AAH00301, XP004370, AAH00614, NP006028, NP005465, NP006035, AAF63491, NP056216, NP057680 and NP060956. Examples of such compounds are antibodies, preferably monoclonal antibodies that specifically react with the histone deacetylase.

Deacetylase inhibitors include, for instance, sodium butyrate, phenylbutyrate and trichostatin A. Particularly preferred are derivatives of said inhibitors showing increased pharmalogical half-life (Brettman and Chaturvedi, J. Cli. Pharmacol. 36 (1996), 617-622). For administration, histone deacetylase may in one embodiment be combined with suitable pharmaceutical carriers. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Administration of the suitable compositions may be effected by different ways, e.g. by intravenous, intraperetoneal, subcutaneous, intramuscular, topical or intradermal administration. The route of administration, of course, depends on the nature of the disease and the kind of compound contained in the pharmaceutical composition. The dosage regimen will be determined by the attending physician and other clinical factors. As is well known in the medical arts, dosages for any one patient depends on many factors, including the patient's size, body surface area, age, sex, the particular compound to be administered, time and route of administration, the kind of the disease, general health and other drugs being administered concurrently.

As used herein, the term “hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid.” The proportion of the population of polynucleotides that forms stable hybrids is referred to herein as the “degree of hybridization”

Hybridization conditions will typically include salt concentrations of less than about 1M, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will hybridize to its target subsequence. Stringent conditions are sequence-dependent and are different under different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the thermal melting point (Tₘ) for the specific sequence at a defined ionic strength and pH. The Tₘ is the temperature (under defined ionic strength, pH and nucleic acid composition) at which 50% of the probes complementary to the target sequence hybridize to the target sequence at equilibrium.

Typically, stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook, Fritsche and Maniatis. “Molecular Cloning A laboratory Manual” 2nd Ed Cold Spring Harbor Press (1989) and Anderson “Nucleic Acid Hybridization” 1st Ed, BIOS Scientific-Publishers Limited (1999), which are hereby incorporated by reference in their entireties for all purposes above.

The term “labeled”, with regard to, for example, a probe, is intended to encompass direct labeling of the probe by coupling (i.e., physically linking) a detectable substance to the probe, as well as indirect labeling of the probe by reactivity with another reagent that is directly labeled Examples of indirect labeling include detection of a primary antibody using a fluorescently labeled secondary antibody and end-labeling of a DNA probe with biotin such that it can be detected with fluorescently labeled streptavidin.

As used herein, “mRNA” includes, but is not limited to, pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, a cRNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

As used herein, the term “nucleic acid”, and equivalent terms such as polynucleotide, refers to a polymeric form of nucleotides of any length, such as ribonucleotides, deoxyribonucleotides or peptide nucleic acids (PNAs), that comprise purine and pyrimidine bases, or other natural, chemically or biochemically modified, non-natural, or derivatized nucleotide bases. The nucleic acid may be double stranded or single stranded. References to single stranded nucleic acids include references to the sense or antisense strands. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. The terms nucleoside, nucleotide, deoxynucleotides and deoxynucleotide generally include complements, fragments and variants of the nucleoside, nucleotide, deoxynucleoside and deoxynucleotide, or analogs thereof.

An “oligonucleotide” as used herein is a single stranded molecule which may be used in hybridization or amplification technologies. In general, an oligonucleotide may be any integer from about 13 to about 100 nucleotides in length, but may also be of greater length.

The terms “polypeptide” and “protein” are used interchangeably and refer to any polymer of amino acids (dipeptide or greater) linked through peptide bonds or modified peptide bonds, whether produced naturally or synthetically. The polypeptides of the invention may comprise non-peptidic components, such as carbohydrate groups. Carbohydrates and other non-peptidic substituents may be added to a polypeptide by the cell in which the polypeptide is produced, and will vary with the type of cell. Polypeptides are defined herein, in terms of their amino acid backbone structures; substituents such as carbohydrate groups are generally not specified, but may be present nonetheless.

The term “patient” refers to human patients or other mammals and includes any individual where it is desirable to examine or treat the patient using the methods of the invention. Suitable mammals that fall within the scope of the invention include, but are not restricted to, primates, livestock animals (eg. sheep, cows, horses, donkeys, pigs), laboratory test animals (eg. rabbits, mice, rats, guinea pigs, hamsters), companion animals (eg. cats, dogs) and captive wild animals (eg. foxes, deer, dingoes).

The term “probe” refers to any molecule which is capable of selectively binding to a specifically intended target molecule, for example, a nucleotide transcript or protein. Probes can be either synthesized by one skilled in the art, or derived from appropriate biological preparations. For purposes of detection of the target molecule, probes may be specifically designed to be labeled. Examples of molecules that can be utilized as probes include, but are not limited to, RNA, DNA, proteins, antibodies, and organic molecules. In some embodiments, a probe can be surface immobilized. Where nucleic acids (such as oligonucleotides) are used they may be capable of binding in a base-specific manner to another strand of nucleic acid. Hybridization may occur between complementary nucleic acid strands or between nucleic acid strands that contain minor regions of mismatch. Such probes include peptide nucleic acids, as described in Nielsen et al., Science 254:1497-1500 (1991); Nielsen Curr. Opin. Biotechnol., 10:71-75 (1999) and other nucleic acid analogs and nucleic acid mimetics.

As used herein, “solid support”, “support”, and “substrate” are used interchangeably and include a reference to a material or group of materials which may have a rigid or semi-rigid surface or surfaces. In many embodiment at least one surface of the solid support will be substantially flat, although in some embodiments it may be desirable to physically separate synthesis regions for different compounds with, for example, wells, raised regions, pins, etched trenches, or the like. According to other embodiments, the solid support(s) may take the form of beads, resins, gels, microspheres, or other geometric configurations. Examples of supports include glass, polystyrene, polypropylene, polyethylene, dextran, nylon, amylases, natural and modified celluloses, polyacrylamides, gabbros, and magnetite. See also U.S. Pat. No. 5,744,305 for exemplary substrates.

As used herein the term “treatment”, refers to any and all methods which remedy a disease state or symptoms, prevent the establishment of disease, or otherwise prevent, hinder, retard, or reverse the progression of disease or other undesirable symptoms in any way whatsoever. The term “treatment” includes, inter alia,: (i) the prevention or inhibition of cancer or cancer recurrence, (ii) the reduction or elimination of symptoms or cancer cells, and (iii) the substantial or complete elimination of the cancer in question. Treatment may be effected prophylactically or therapeutically. Treatment may entail treatment with a single agent or a combination (more than two) of agents. An “agent” is used herein broadly to refer to, for example, a compound or other means for treatment e.g. radiation treatment or surgery.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to6 etc., as well as individual numbers within that range, for example, 1, 2, 3, 4, 5, and 6. This applies regardless of the breadth of the range.

Unless otherwise indicated, all technical and scientific terms used herein have the same meaning as commonly understood to one of ordinary skill in the art to which the invention belongs.

### BRIEF DESCRIPTION OF DRAWINGS

FIG. 1. HDACIs SAHA and TSA promote E2F1-mediated cell death (a) p53 null HCT116-ER-E2F1 expressing cells or control ER expressing cells were treated with or without 4-OHT. Cyclin E and p73 expression were evaluated by immunoblot analysis. (b) ER-E2F1 expressing or control cells were treated with 1 μM SAHA (left panel) or 100 nM TSA (right panel), in the presence or absence of 4-OHT. After 48 h, cells were harvested and cell death was assessed by PI staining using FACS. Mean results of three independent experiments were shown with standard deviations. (c) Colony formation assay. 1000 ER-E2F1 expressing cells and control cells were plated per well of 6-well plate for 48 h, and followed by indicated treatment for 24 h. Cells were washed and fresh DMEM was added, colonies were stained with crystal violet 15 days later. Representative plates are shown. (d) Saos-2 and HCT116 cells infected with adenovirus containing E2F1 or LacZ as indicated were treated with 100 nM TSA for 24 h, and cell death was analyzed by FACS.

FIG. 2. HDACIs selectively activate E2F1 target genes. (a) ER-E2F1 expressing cells were treated with 100 nM TSA for indicated times or 1 μM SAHA for 24 h in the presence or absence of 4-OHT. mRNA levels of E2F1 target genes as well as p21 and GAPDH were detected with RT-PCR. (b) Cells were treated in (a). The expressions of E2F1 target gene products were assessed by Western blot with antibodies to Bim, Puma, caspase-3 and p73. (c) Saos-2 and IMR90 cells were infected with Ad-E2F1 or Ad-LacZ for 24 h before treatment with 100 nM TSA for additional 24 h Expressions of Bim, p73, Puma and E2F-1 were assessed by Western blot with corresponding antibodies.

FIG. 3. The effect of Bim-specific siRNA on HDACI-induced apoptosis upon E2F1 overexpression. (a) ER-E2F1 were transfected with nonspecific control siRNA (NC siRNA) or Bim-specific siRNA for 48 h, and then either left untreated (−) or treated with 4-OHT, SAHA or both for additional 24 h. The expression of Bim was analyzed by Western blotting (left panel). Cell death was analyzed by flow cytometry following propidium iodide staining (right panel). The percentages of cells in Sub-G1 are indicated. (b) ER-E2F1 were transfected with nonspecific control siRNA (NC siRNA) or Bim-specific siRNA for 48 h, and then either left untreated (−) or treated with 4-OHT, 100 nM TSA or both for additional 48 h. The expression of Bim was analyzed by Western blotting (left panel). Cell death was assessed as in (a) and the graph shows the mean results of three independent experiments with standard deviations (right panel). (c) Saos-2 cells treated with Bim or control siRNA were infected with Ad-E2F1 or Ad-LacZ for 24 h and followed by 100 nM TSA treatment for additional 24 h. Bim expression was analyzed by Western blotting (left panel) and cell death was assessed as in (a) and the graph shows the mean results of three independent experiments with standard deviations (right panel).

FIG. 4. SAHA promotes E2F1 recruitment to the Bim promoter. (a) Schematic representation of human Bim promoter containing putative, E2F-binding sites. The indicated regions were isolated and cloned into pGL3 reporter construct. (b) pGL3-basic, Bim −1415/−205 or −2415/−1333 luciferase construct and renillia luciferase construct were transfected into HCT116 cells together with increasing amounts of E2F1 expressing vector (0, 20 and 40 ng). Relative luciferase activities were measured 48 h after transfection. Results are depicted as fold induction, after normalization to the renillia luciferase activity. Data shown represent the average of three independent experiments, and the error bars show the standard deviation. (c) Left panel, schematic representation of the human Bim promoter containing the E2F1-RE element. Right panel, E2F1 binding to the Bim promoter was analyzed by ChIP. Crosslinked chromatin from ER-E2F1 expressing cells treated as indicated were immunoprecipitated with antibody to E2F1, non-specific IgG, and then Bim promoter fragment was amplified by PCR using primers flanking the E2F1-RE containing region. Positive control (input DNA) amplifications are shown.

FIG. 5. Rb inactivation enhances Bim expression and sensitizes HDACIs-induced apoptosis. (a) U2OS cells were infected with Ad-E1A or Ad-LacZ for 24 h and treated with SAHA (1 μM) for additional 48 h. Cell death was determined by FACS analysis (right panel), and the levels of Bim ard p73 were assessed by immunoblotting using antibodies against the indicated proteins (left panel). (b) Normal IMR90 and transformed IMR90-E1A cells were treated with SAHA at indicated concentrations and cell proliferation was evaluated for indicated times. (c) Expression of E1A results in upregulation of Bim and apoptosis potentiation in response to SAHA and TSA. IMR90 and IMR90-E1A cells were treated with SAHA (2.5 μM) and TSA (300 nM) for 48 h. Cell death was determined by FACS analysis (left panel), and the levels of Bim and p73 were assessed by immunoblotting using antibodies against indicated proteins (right panel). (d) IMR90-E1A cells transfected with Bim siRNA or control siRNA (NC siRNA) were treated with TSA (800 nM) for 24 h. The expressions of Bim and β-actin were assessed by immunoblotting using antibodies against the indicated proteins. Apoptosis was evaluated by FACS analysis. (e) Saos-2 cells were transfected with E2F1 siRNA or negative control siRNA (NC siRNA) and treated with TSA (150 nM) or SAHA (1 μM). The expressions of E2F1, Bim and PARP were assessed by immunoblotting using antibodies against the indicated proteins. NS, non-specific band as the loading control. Apoptosis was evaluated by FACS analysis.

FIG. 6. Cell death response and expression analysis of apoptosis genes associated with SAHA and E2F1. (A). ER-E2F1-expressing or control ER-expressing cells were treated with 1 μM SAHA in the presence or absence of 4-OHT. After 48 h, cells were harvested, and stained for active anti-caspase-3. Percentages of cells positive for active caspase-3 are indicated. (B). E2F1-regulated apoptosis genes. Microarray analysis as illustrated in Cluster and Tree Viewer showing E2F1-dependent genes in ER-E2F1 and ER cells treated with 4-OHT. Red represents up-regulation relative to the untreated control (black). (C). SAHA-responsive genes in ER-E2F1 cells in the presence or absence of 4-OHT. Genes in boxes are putative E2F1 targets identified in B.

FIG. 7. E2F1 induces the ASK1 mRNA and protein accumulation. (A). p53 null HCT116 cells were infected with an empty retrovirus (−), a retrovirus expressing ER-E2F1 wild-type (wt) or ER-E2F1-E132 (E132) cells were left untreated (−) or treated with OHT for the indicated duration. ASK1 mRNA and protein expression levels were analyzed by RT-PCR (left panel) and Western blot (right panel), respectively. (B) U-2OS cells had been synchronized in G0 (0 h) by serum starvation for 48 h and then reentered the cell cycle after serum stimulation. The corresponding cell cycle distribution is shown (left panel). Proteins were extracted from cells at different time after release into the cell cycle. E2F1, ASK1 and cyclin E were analyzed by Western blotting (right panel). (C). Western blot analysis of ASK1, p73 and α-tubulin protein in U2OS and IMR90 cell infected with control adenovirus (−) or an adenovirus expressing E1A (+).

FIG. 8. E2F1 binds to and activates ASK1 promoter. (A) Schematic representation of human ASK1 promoter. Putative E2F-binding sites, and the deletion constructs used in this study, are indicated. (B) HCT116 cells were transfected with the PGL3-basic, E2F1 (50 or 100 ng), together with a luciferase reporter construct containing the ASK1 promoter (−1000/+125). Relative luciferase activities were measured 48 h after transfection. Results are depicted as fold induction, after normalization to the Renillia luciferase activity. (C) Mapping of the E2F1 DNA-binding region. HCT116 cells were transfected with luciferase reporter constructs containing the indicated ASK1 promoter deletions and 100 ng of E2F1. (D) ER-E2F1 and ER-E132 expressing cells were treated with or without 4-OHT for 16 h. ChIP assay was performed using anti-E2F1 antibody or non-specific IgG. ASK1 promoter region from −273 to +125 was amplified by PCR.

FIG. 9. ASK1 regulates E2F1 target gene expression through Rb inactivation. (A) ER-E2F1 expressing cells were transfected with non-specific control siRNA (NC siRNA) or ASK1-specific siRNA for 48 h, and then either left untreated (−) or treated with 4-OHT, The expression of ASK1, Bim, Cyclin E and p73 were analyzed by Western blotting. (B) HCT116 cells were co-transfected with a luciferase reporter plasmid containing the Bim promoter, together with E2F1, Rb or ASK1 expression vector. In addition, ASK1 expression plasmid was cotransfected with E2F1 to determine the effect of ASK1 on E2F1-mediated activation of Bim promoter in the presence or absence of Rb overexpression.

FIG. 10. SAHA promotes E2F1-mediated ASK1 induction. (A) ER-E2F1 expressing cells were treated with 1 μM SAHA for 24 h in the presence or absence of 4-OHT. The ASK1 and Bim protein levels were assessed by Western blotting. (B) ER-E2 F1 expressing cells were treated as (A) and analyzed by ChIP using E2F1 antibody. PCR amplification products of E2F1-ChIP using ASK1 promoter primers were analyzed by agarose gel electrophoresis. (C) IMR90 and IMR90-E1A cells were treated with SAHA for 24 h. The levels of ASK1 and α-tubulin were assessed by Western blotting. (D) U2OS cells were treated with 2.5 μM SAHA for indicated times. The expression of ASK1 was analyzed by western blotting (left panel). U2OS cells were transfected with NC siRNA and E2F1 siRNA for 24 hours and treated as (C). The levels of E2F1, ASK1 and the α-tubulin were assessed by Western blotting (left panel).

FIG. 11. Suppression of ASK1 expression inhibits SAHA-induced apoptosis upon E2F1 activation. (A) ER-E2F1 expressing cells were transfected with NC siRNA and ASK1 siRNA and treated with SAHA in the presence or absence of 4-OHT. Cell death was determined by FACS analysis. Mean results of three independent experiments were shown with standard deviations. (B) Cells were treated in (A). The levels of ASK1, Bim, phospho-p38, p38, phospho-JNK, JNK were analyzed by Western blotting.

### DETAILED DESCRIPTION

The inventors have discovered that cells with increased E2F1 activity or Rb inactivation are highly susceptible to HDACI-induced cell death tumors with a deregulated Rb-E2F1 pathway.

Accordingly, a first aspect of the invention provides a method of assessing the suitability of a cancer patient for treatment with a histone deacetylase inhibitor, the method comprising assaying a biological sample from the patient for elevated E2F1 activity.

The assay may be based on, for example, measurement of expression of E2FP target genes (such as ccne1 and ccne2), or increased Cdk4 expression that can result in elevated E2F1 activity. The results of the assay may then be used (optionally in conjunction with other data etc.) to assign an appropriate treatment regime to the patient. As discovered by the inventors of the present application, elevated E2F1 levels indicate that the cancerous cells are likely to be sensitive to HDACI and for such patients HDACI may accordingly be appropriate. Additional cancer treatments may also be selected for such patients, including treatment with other anticancer agents, radiotherapy etc.

Conversely, patients with biological samples having E2F1 activity in the normal range may be considered as patients for whom HDACI treatment would not be considered appropriate as for such patients HDACI is less likely to be effective.

A second aspect of the invention provides selecting a cancer patient for treatment with a HDACI, the method comprising selecting a patient who has assayed positive for elevated E2F1 activity.

A third aspect of the invention provides a method of treating a cancer patient with a HDACI where in the patient's cancer has assayed positive for elevated E2F1 activity.

A fourth aspect of the invention provides for the use of a HDACI in the manufacture of a medicament for the treatment of a cancer patient whose cancer has assayed positive for elevated E2F1 activity.

A fifth aspect of the invention provides for a kit for use in a method of any of the first, second, third or fourth aspects of the invention. The kit comprises one or more reagents for use in assessing E2F1 activity in a biological sample.

In one embodiment the kit comprises one or more components selected from the group consisting of:
(a) a labelled compound or agent capable of detecting a marker protein or nucleic acid in a sample;
(b) means for determining the level of the marker protein or marker nucleic acid in the sample (e.g., an antibody which binds the protein or a fragment thereof, or an oligonucleotide probe which binds to DNA or mRNA encoding the protein);
(c) instructions for interpreting the results obtained using the kit;
(d) a buffering agent;
(e) a preservative;
(f) a protein stabilizing agent;
(g) components for use in detecting the detectable label (e.g., an enzyme or a substrate); and
(h) software, for example software for selecting patient treatment.

It is envisaged that the methods of the invention may find utility in relation to various cancer patients and various types of cancer. For instance, the cancer may, in one embodiment, be selected from the group consisting of: retinoblastoma, breast cancer, lung cancer (e.g. non-small lung cancer or small cell lung cancer), salivary gland tumor, pancreatic cancer, glioblastoma multiforma and mantle cell lymphoma.

Other examples of cancer where the invention may find utility may include: skin cancer, bone cancer, prostate cancer, liver cancer, lung cancer, brain cancer, cancer of the larynx, gallbladder, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuronms, intestinal ganglioneuromas, hyperplastic corneal nerve tumor, marfanoid habitue tumor, Wilm's tumor, seminoma, ovarian tumor, leiomyomater tumor, cervical dysplasia and in situ carcinoma, neuroblastoma, soft tissue sarcoma, malignant carcinoid, topical skin lesion, mycosis fungoide, sarcomas such as rhabdomyosarcoma and Kaposi's sarcoma, osteogenic and other malignant hypercalcemia, renal cell tumor, polycythermia vera, adenocarcinoma, leukemias, lymphomas, malignant melanomas, epidermoid carcinomas, and other carcinomas and sarcomas. Examples of lymphomas include, for example, small lymphocytic lymphoma, follicular lymphoma, large B-cell lymphoma, T-cell lymphoma, and Burkitt lymphoma.

The biological sample which may be assayed includes tissues, cells, body fluids and isolates thereof etc., isolated from the cancer patient, as well as tissues, cells and fluids etc. present within a subject (i.e. the sample is in vivo). Thus, in vivo and in vitro methods are envisaged in the present invention.

One or more biological samples may be employed in the methods of the present invention. Thus assays may be performed on multiple samples from the cancer patient.

Examples of samples include: whole blood, blood fluids (e.g. serum and plasma), lymph and cystic fluids, sputum, stool, tears, mucus, hair, skin, ascitic fluid, cystic fluid, urine, nipple exudates, nipple aspirates, sections of tissues such as biopsy and autopsy samples, frozen sections taken for histologic purposes, archival samples, explants and primary and/or transformed cell cultures derived from patient tissues etc.

In one embodiment the sample may be a “breast-associated” body fluid, which is a fluid which, when in the body of a patient, contacts or passes through breast cells or into which cells, nucleic acids or proteins shed from breast cells are capable of passing. Examples of breast-associated body fluids include blood fluids, lymph, cystic fluid, and nipple aspirates.

Prior to being assayed, the sample may be untreated, treated, diluted or concentrated from a patient.

Persons skilled in the art will appreciate that various methods may be used to assay for elevated E2F1 activity, for example by measurement of expression of E2F1 target genes (such as ccne1 and ccne2) or Cdk4.

E2F1 activity can, for instance, be assessed by measuring (qualitatively or quantitatively) the expression level of at least one gene whose expression (e.g. at the mRNA or protein level) is indicative of E2F1 activity. Preferably, the expression level of multiple (e.g. at least 2, 3, 4, 5, 8, 10, or 15) genes is measured.

As mentioned above, inactivation of Rb results in increased E2F1 activity. Hence, one method of assessing E2F1 activity would be to assess Rb levels or activity. Other markers which are positively or negatively correlated with E2F1 activity may alternatively or additionally be assayed in order to provide an indication of E2F1 activity. Examples of markers whose expression may be correlated with E2F1 activity include: the E1A, p16(INK4), cyclin D, Cdk4, Cdk6, cyclin E1, cyclin E2.

The level of a marker may be determined by any means known in the art. The level may be determined by, for example, determining the level of nucleic acid transcribed from a marker gene. Alternatively, or additionally, the level of specific proteins translated from mRNA transcribed from a marker gene may be determined. In yet another embodiment, the level of a metabolite which is produced directly (i.e., catalyzed) or indirectly or “consumed” by the corresponding marker protein could be determined.

An exemplary assay for determining the level of a marker involves obtaining a sample of an individual and contacting the sample with a probe (e.g. antibody, oligonucleotide) capable of detecting the marker protein or marker nucleic acid (e.g., mRNA, genomic DNA, or cDNA) under appropriate conditions and for a time sufficient to allow the marker and probe to interact and bind, thus forming a complex that can be removed and/or detected in the reaction mixture. The detection methods of the invention can thus be used to detect mRNA, protein, cDNA, or genomic DNA, for example, in a biological sample in vitro as well as in vivo.

These assays can be conducted in a variety of ways. For example, one method to conduct such an assay would involve anchoring the marker or probe onto a solid support and detecting target marker/probe complexes anchored on the solid phase at the end of the reaction. In one embodiment of such a method, a sample of an individual, which is to be assayed for presence, amount and/or concentration of marker, can be anchored onto a solid support. In another embodiment, the reverse situation is possible, in which the probe can be anchored to a solid support (e.g. a nylon membrane or a chip) and a sample of an individual can be allowed to react as an unanchored component of the assay. In one embodiment, the probes may be immobilized on a microarray.

In order to conduct assays with the above-mentioned approaches, the non-immobilized component is added to the solid support upon which the second component is anchored. After the reaction is complete, uncomplexed components may be removed (e.g., by washing) under conditions such that any complexes formed will remain immobilized upon the solid support. The detection of marker/probe complexes anchored to the solid support can be accomplished in a number of methods.

In a preferred embodiment, the probe, when it is the unanchored assay component, can be labeled for the purpose of detection and readout of the assay, either directly or indirectly, with a detectable label. It is also possible to directly detect marker/probe complex formation without further manipulation or labeling of either component (marker or probe), for example by utilizing the technique of fluorescence energy transfer (see, for example, Lakowicz et al., U.S. Pat. No. 5,631,169; Stavrianopoulos, et al., U.S. Pat. No. 4,868,103).

The level of expression of specific marker genes can, for example, be accomplished by d mining the amount of mRNA (or polynucleotides derived therefrom) present in a sample.

Many techniques for the detection and quantification of mRNA levels involve contacting the mRNA with a nucleic acid molecule (probe) that can hybridize to the mRNA (or polynucleotide derived therefrom). The nucleic acid probe can be, for example, a polynucleotide of at least 7, 10, 15, 17, 18, 20, 25, 30, 40, 50, 100 nucleotide residues in length. Probes may include, but are not limited to, oligonucleotides, cDNA, or RNA. Probes may contain a detectable label, such as a fluorescent or chemiluminescent label. When a method of assessing marker expression is used which involves hybridization of one nucleic acid with another, it is preferred that the hybridization be performed under stringent hybridization conditions.

Methods for conducting polynucleotide hybridization assays have been well developed in the art Hybridization assay procedures and conditions will vary depending on the application and are selected in accordance with the general binding methods known including those referred to in: Maniatis et al. Molecular Cloning. A Laboratory Manual (2^{nd} Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davis, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The present invention contemplates signal detection of hybridization between ligands in certain preferred embodiments. See U.S. Pat. Nos. 5,143,854; 5,578,832; 5,631,734; 5,834,758; 5,936,324; 5,981,956; 6,025,601; 6,141,096; 6,185,030; 6,201,639; 6,218,803; and 6,225,625, U.S. Patent Application No. 60/364,731 and PCT Application No. PCT/US99/06097 (published as WO99/47964), each of which also is hereby incorporated by reference in its entirety for all purposes.

Methods and apparatus for signal detection and processing of intensity data are disclosed in, for example, U.S. Pat. No. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent Application 60/364,731 and in PCT Application PCT/US99/06097 (published as WO99/47964), each of which also is hereby incorporated by reference in its entirety for all purposes.

Any method for determining nucleic acid or protein levels can be used in the present invention and the examples described herein are not intended to be limiting.

In one format, mRNA is immobilized on a solid support and contacted with a probe, for example by running the isolated mRNA on an agarose gel and transferring the mRNA from the gel to a solid support such as a filter. Nucleic acid probes representing one or more markers are then hybridized to the filter by northern hybridization, and the amount of marker-derived RNA is determined. Such determination can be visual, or machine-aided, for example, by use of a densitometer. In an alternative format, the probe(s) are immobilized on a solid-support and the nucleic acid is contacted with the probe(s), for example, in an Affymetrix gene chip array.

The present invention also contemplates sample preparation methods in certain preferred embodiments. For example, prior to or concurrent with gene expression analysis, the sample may be amplified by a variety of mechanisms, some of which may employ amplification techniques such as PCR (e.g. RT-PCR) and the ligase chain reaction (LCR) etc. The sample may be amplified on the array. See, for example, U.S. Pat. No. 6,300,070 and U.S. patent application Ser. No. 09/513,300, which are incorporated herein by reference.

In one embodiment of the present invention, the level of a marker protein is determined. A preferred agent for determining the level of a marker protein of the invention is an antibody capable of binding to such a protein or a fragment thereof, preferably an antibody with a detectable label.

Suitable antibodies can be produced using techniques well known to those of skill in the art and disclosed in, for example, U.S. Pat. Nos. 4,011,308; 4,722,890; 4,016,043; 3,876,504; 3,770,380; and 4,372,745. Preferably, monoclonal antibodies are employed. Monoclonal antibodies are generally prepared using the method of Kohler & Milstein (1975) Nature 256:495-497, or a modification thereof.

A variety of formats can be employed to determine whether a sample contains a protein that binds to a given antibody. Examples of such formats include, but are not limited to, enzyme immunoassay (EIA), radioimmunoassay (RIA), enzyme linked immunosorbent assays (ELISAs), Western blots, immunoprecipitations and immunofluorescence. In such uses, it is generally preferable to immobilize either the antibody or proteins on a solid support. Suitable supports include any support capable of binding an antigen or an, antibody. In one embodiment, marker derived in levels can be determined by constructing an antibody microarray in which binding sites comprise immobilized, preferably monoclonal, antibodies specific to a marker protein. By utilising antibodies which are specific for different marker proteins, the level of more than one marker protein may be determined using a single microarray.

In addition, preferred in vivo techniques for detection of a marker protein include introducing into a subject a labeled antibody directed against a marker protein. For example, the antibody can be labeled with a radioactive marker whose presence and location in a subject can be detected by standard imaging techniques.

The present invention can employ solid supports, including arrays in some preferred embodiments. Methods and techniques applicable to polymer (including protein) array synthesis have been described in numerous publications and as such should pose no problem for the skilled person.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques may be applied to polypeptide (e.g. antibody) arrays.

In preferred embodiments, polynucleotide microarrays are used to determine the level of a marker. In this way, the expression status of more than one marker may be assessed simultaneously.

Microarrays may be prepared by selecting probes which comprise a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic DNA. The polynucleotide sequences of the probes may also be synthesized nucleotide sequences, such as synthetic oligonucleotide sequences. The probe sequences can be synthesized either enzymatical in vivo, enzymatically in vitro (eg., by PCR), or nonenzymatically in vitro.

A skilled artisan will also appreciate that it may be desirable to include positive control probes, e.g., probes known to be complementary and hybridizable to sequences in the target polynucleotide molecules, and/or negative control probes, e.g., probes known to not be complementary and hybridizable to sequences in the target polynucleotide molecules, on the array.

The present invention may make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

In addition to assessing E2F1 activity other factors may also be taken into account when selecting treatment e.g. gender, age, previous cancer history, benign breast disease, hereditary factors (family history of cancer), obesity, low physical activity, use of postmenopausal hormone replacement therapy, use of oral contraceptives, exposure to ionizing radiation, dietary practices, or alcohol consumption.

In one aspect of the invention there is provided a kit. The kit comprises one or more reagents for use in assessing E2F1 activity in a biological sample. The kit may be promoted, distributed, or sold as a unit for performing a method of the present invention.

The kit can comprise a labeled compound or agent capable of detecting a marker protein or nucleic acid in a sample and means for determining the level of the marker protein or marker nucleic acid in the sample (e.g., an antibody which binds the protein or a fragment thereof, or an oligonucleotide probe which binds to DNA or mRNA encoding the protein). Kits can also include instructions for interpreting the results obtained using the kit.

For antibody-based kits, the kit can comprise, for example: (1) a first antibody (e.g., attached to a solid support) which binds to a marker protein, and, optionally, (2) a second, different antibody which binds to either the marker protein or the first antibody and which is optionally conjugated to a detectable label.

For oligonucleotide-based kits, the kit can comprise, for example: (1) an oligonucleotide, e.g., a detectably labelled oligonucleotide, which hybridizes to a nucleic acid marker and/or (2) a pair of primers useful for amplifying a marker nucleic acid molecule. The kit can also comprise, e.g., one or more of the following: a buffering agent, a preservative, or a protein stabilizing agent. The kit can further comprise one or more components for use in detecting the detectable label (e.g., an enzyme or a substrate).

The kit can also contain a control sample or a series of control samples which can be assayed and compared to the test sample. Each component of the kit can be enclosed within an individual container and all of the various containers can be within a single package, along with instructions for interpreting the results of the assays performed using the kit.

The kit of the invention may optionally comprise additional components useful for performing a method of the invention. By way of example, the kit may comprise fluids (e.g., SSC buffer) suitable for annealing complementary nucleic acids or for binding an antibody with a protein with which it specifically binds, one or more sample compartments, an instructional material which describes performance of a method of the invention, and the like.

In one embodiment, the kit may comprise a microarray, e.g. an oligonucleotide microarray or an antibody microarray.

In one embodiment the kit comprises software, for example software for selecting patient treatment. Such software might include instructions for the computer system's processor to receive data structures that include the level of expression various markers which may be correlated with E2F1 activity and optionally also clinical information about the patient, e.g. the patient's age etc.

By “elevated E2F1 activity” we include where the activity of E2F1 is higher than a normal level of E2F1 activity. A “normal level” of E2F1 activity includes the level of E2F1 activity in a non-cancerous or benign sample. In one embodiment, the E2F1 activity in a biological sample from a cancer patient may be compared with a mean, median, or mode level of E2F1 activity in non-cancerous or benign sample.

The level of E2F1 activity in the biological sample from the cancer patient may be assessed qualitatively or quantitatively. A qualitative or quantitative comparison with a normal level of E2F1 activity can then be carried out.

When assessing the level of E2F1 activity in a biological sample from a cancer patient the level of E2F1 activity in one or more positive or negative controls (e.g. benign or non-cancerous samples) may also be assessed. The one or more controls may comprise data obtained at the same or similar time as the patient's individual data, or may be a stored value or set of values e.g. stored on a computer, or on computer-readable media.

In one embodiment a quantitative assessment of E2F1 activity may be performed. The level of E2F1 activity may in one embodiment be considered as being elevated where the level is greater than a pre-determined cut-off level. In one embodiment, the pre-determined cut-off level is at least 10%, 30%, 50%, 80%, 100%, 150%, 200%, 150%, 300% greater relative to a mode, median or mean level of E2F1 activity of benign cells or normal tissue. In one embodiment, the pre-determined cut-off level is chosen so as to have a statistically significant p-value (e.g. a p-value of less than 0.05) for the level of E2F1 activity as compared with normal E2F1 activity levels.

In addition to assessing whether there is elevated E2F activity, further tests may be carried out. Such further tests may yield further data regarding the cancer. Such further data may for instance be of assistance in selecting an appropriate treatment regime for the patient. The one or more further tests may be carried out on the one or more biological samples which are assessed for elevated E2F activity or one or more different biological samples.

Various HDACIs are known in the art and HDACIs include a range of compounds including: short-chain fatty acids (eg butyrate), hyroxamic acids (eg SAHA & Trichostatin), epoxyketones (eg trapoxin), benzamides, and a variety of other miscellaneous chemical families. Examples of HDACIs which may be employed include: tricostatin A (TSA), suberoylanilide hydroxamic acid (SAHA), phenylbutyrate, scriptaid, apicidin, pyroxamide, depsipeptide, pivaloyloxymethylbutyrate (also known as AN-9); cyclostellettamine, particularly cyclostellettamine A, cyclostellettamine, G dehydrocyclostellettamine D and dehydrocyclostellettamine E. Further examples of HDACIs will be known to those skilled in the art and may also be employed in the present invention. For instance, HDACIs and details of how they may be employed are disclosed in: WO05105066, WO05105055, WO05097747, WO05092899, WO05066151, WO05059167, WO05055928, WO05030705, WO05030704 and WO05030239.

The practice of the present invention will employ, unless otherwise indicated, conventional techniques of molecular biology, microbiology, and recombinant DNA technology which are within the skill of those working in the art. Such techniques are explained fully in the literature. Examples of texts for consultation include the following: Sambrook Molecular Cloning; A Laboratory Manual, Third Edition (2000).

In order that the invention may be readily understood and put into practical effect, particular preferred embodiments will now be described by way of the following non-limiting examples.

### MODES FOR CARRYING OUT THE INVENTION

### Examples

### Experimental Procedure

Cell Culture and Chemicals

p53 null colon cancer HCT116 cells were kindly provided by Dr. Bert Vogelstein (Johns Hopkins University, MD). Normal human lung fibroblast cells IMR90, Osteosarcoma U2OS and Saos-2 cells were from ATCC. Transformed IMR90-E1A cells were kindly provided by Dr. Claudio Brancolini (University of Di Udine, Italy). All cell culture reagents and media were from Invitrogen. TSA was purchased from Cell Signaling Technologies and SAHA was from Alexis Biochemicals (San Diego, Calif.). To generate E2F1 overexpressing cells, 293 cells with transfected with pBabe.Haemagglutinin epitope (HA)ER or pBabe.HA.ER-E2F1 expression vectors (30), and viral supernatants were used to infect p53 null HCT116 cells. Infected cells were selected with 2 μg/l puromycin, and individual clones were isolated and expanded under selection conditions. To activate ER-E2F1, 1-3 μM of 4-hydroxytamoxifen (4-OHT) was added to the tissue culture medium.

Microarray hybridization and data analysis. Total RNA was extracted with the use of Trizol reagent (Invitrogen, Carlsbad, Calif.) and the Qiagen RNAease Mini kit according to the manufacture's instructions (Valencia, Calif.). For all experiments, universal human reference RNA (USR) (Stratagene, La Jolla, Calif.) was used to generate a reference probe for drug treated and untreated samples. 30 μg of total RNA from experimental samples or equal amount of UHR were labeled with Cy5 and Cy3, respectively, by using Superscript II Reverse Transcriptase (Invitrogen, Carlsbad, Calif.). The microarray hybridization, image process, and data normalization were as described previously (61). The log 2 ratios of each time point were then normalized for each gene to that of untreated cells (time 0) to obtain the relative expression pattern. Gene expression values of 220 apoptosis-related genes were extracted and were analyzed using clustering and display programs (rana.stanford.edu/software) developed by Eisen et al (62).

Adenoviral Infections

Adenovirus Ad-E2F1 was obtained from Dr. Joseph Navins (Duke University, Durham, N.C.) and Ad-E1A was from Dr. Andrew Turnell (University of Birmingham, Birmingham, UK). Cells were grown to 50% confluence and infected with recombinant adenovirus. Twenty-four hours after the infection, cells were treated, with drugs for indicated times.

Flow Cytometry Analysis of DNA Content

Cells were harvested and fixed in 70% ethanol. Fixed cells were stained with propidium iodide (50 μg/ml) after treatment with RNase (100 μg/ml). The stained cells were analyzed for DNA content by fluorescence-activated cell sorting (FACS) in a FACScalibur (Becton Dickinson Instrument, San Jose, Calif.). Cell cycle fractions were quantified using the CellQuest software (Becton Dickinson).

Caspase Activity

To measure caspase-3 activity, cells were fixed with Cytofix/Cytoperm solution (BD PharMingen) as instructed, and then stained with FITC-conjugated rabbit anti-active caspase-3 monoclonal antibody (BD PharMingen). Quantification of cells positive for the caspase-3 detection was performed by flow cytometry.

Colony Formation Assay

1000 cells were plated per well of a 6-well plate for 24-48 h and then followed by 100 nM TSA or 1 μM SAHA treatment in the presence or absence of 4-OHT. After 24 h, the drugs were removed and replaced with fresh medium. Colonies were stained with crystal violet and counted after 12 days.

Western Blotting

Cells were scraped, collected and lysed. Protein samples (50 μg) were separated by SDS/PAGE and transferred onto immobilon membranes (Millipore, Bedford, Mass.). Antibodies against the following proteins were used: E2F1, ASK1, p73, PARP, Cyclin E, α-Tubulin, and β-actin (Santa Cruz Biotechnology), Bim (BD Pharmingen), Caspase-3 (Cell Signaling Technologies), phospho-p38, p38, phospho-JNK, JNK (Cell Signaling Technologies). Signals were detected by enhanced chemiluminescence signal by X-ray film (Kodak, Rochester, N.Y.).

Reverse Transcription-PCR

One microgram of total RNA from each sample was subjected to PCR with reverse transcription using the One Step RT-PCR kit (Clontech) according to the manufacturer's protocol. Selected genes were analysed for PCR analysis. PCR was carried out for 20-30 cycles, with each cycle consisting of a denaturing step for 1 min at 94° C., an annealing step for 2 min at 58° C., and a polymerization step for 2 min at 72° C. The PCR product was separated on a 2.0% agarose gel containing ethidium bromide and photographed under ultraviolet light. The primer sequences are available upon request.

Luciferase Reporter Assay

Genomic DNA encompassing the human Bim promoter elements −1415/−205 and −2415/−1333, and the human ASK1 promoter elements −1000/+125, −1000/−256 and −273/+125 were cloned into pGL3-Luciferase construct (Promega, Madison, Wis.). Luciferase assays were performed using the Dual Luciferase system (Promega). HCT116 cells were plated at a density of 5×10⁴ cells per well of a 24-well plate. Bim promoter luciferase constructs and a control construct were transfected into HCT116 cells with E2F1, ASK1 or Rb expression vector. Twenty-four hours after transfection, the luciferase activities were analysed using the Dual Luciferase system.

Chromatin Immunoprecipitation (ChIP)

CHIP assays were performed as described previously for E2F1(31). Briefly, ER-E2F1 expressing cells treated with SAHA in the presence or absence of 4-OHT were crosslinked with 1% formaldehyde for 10 min at room temperature. Formaldehyde was inactivated by addition of 125 mM, Glycine. Chromatin extracts containing DNA fragment of average size of 500 bp were immunoprecipitated using anti-E2F1 polyclonal antibody (C20, Santa Cruz). The DNA was extracted by Phenol:Chlorofbrm:IAA (Ambion). The DNA recovered was subjected to amplification by PCR using HotStarTaq Master fix Kit (Qiagen). Approximately 20 ng of the input and the ChIP samples were used as template in each reaction. Reaction mixtures were initially melted at 95° C. for 15 min followed by 27 cycles of 94° C./30 sec, 60° C./30 sec, 72° C./30 sec, and a final extension of 72° C. for 5 min. The primer sequences are available upon request PCR primers for Bim are 5′-GCTGCTAAGGCTTGTGTCCGGA-3′ (forward) and 5′-TGCCCGCGTTCCCAATTGGT-3′ (reverse).

RNA Interference

Bim specific siRNA and negative control siRNA were purchased from Cell Signaling Technologies. SMARTpool® E2F1 siRNA, ASK1 siRNA and negative control siRNA were purchased from Dharmacon, Inc. (Lafayette, Colo.). Cells were transfected with Lipofectamine 2000 according to the manufacturer's protocol in the presence of siRNAs.

Results

E2F1 Overexpression Facilitates HDACIs-Induced Cell Death in Human Cancer Cells.

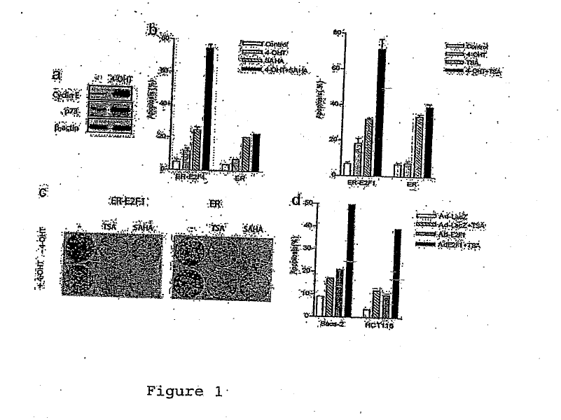

Activation of E2F1 induces apoptosis through both p53-dependent and independent mechanisms. The former is primarily mediated through the p19^{ARF}/Mdm2 pathway and has been well-characterized (24, 25, 32, 33). To investigate the regulation of E2F1 apoptotic activity independent of p53, we used p53 null HCT116 cells to establish cell line stably expressing E2F1 fused to the 4-hydroxytamoxifen (4-OHT)-responsive ligand-binding domain of the estrogen receptor (ER)(30). ER-E2F1 fusion protein is maintained inactive in the cytoplasm in the absence of 4-OHT and becomes activated after addition of 4-OHT by allowing ER-E2F1 translocation to the nucleus (30, 34). As expected, addition of 4-OHT in ER-E2F1 expressing cells led to a strong increase of cyclin E and p73 expression (FIG. 1a), two bona fide E2F1 targets (26, 27, 35).

To investigate whether E2F1 activation induces sensitization of cells to HDACIs-induced cell death, we analyzed the cell cycle profiles by FACS (fluorescence-activated cell sorter) in ER-E2F1 expressing HCT116 p53 null cells (ER-E2F1) and the control cells expressing-ER-binding domain only (ER) following treatment with the HDACIs SAHA or TSA in the presence or absence of 4-OHT. In ER-E2F1 expressing cells, 4 OHT or SAHA alone induced approximately 13% and 23% of sub-G1 cells indicative of cell death, respectively. Addition of both 4-OHT and SAHA resulted in a marked increase in sub-G1 cells (75%), indicating a strong synergistic cell death response (FIG. 1b, left panel). This synergistic effect was however not seen in the control ER expressing cells. Identical results were obtained in TSA treated cells (FIG. 1b, right panel). Moreover, E2F1 activation by 4-OHT greatly promoted TSA or SAHA-induced growth inhibition, as determined by colony formation assay (FIG. 1c). These results indicate that E2F1 overexpression resulted in enhanced cell death and growth inhibition in response to HDACIs.

To determine the effects of E2F1 overexpression on HDACIs-induced apoptosis using a different system and an additional cell type, we infected p53-deficient osteosarcoma Saos-2 cells and p53 normal HCT116 cells with either E2F1 expressing adenovirus (Ad-E2F1) or control adenovirus (Ad-LacZ) and monitored the cell death response after TSA treatment. As shown in FIG. 1d, in each case, cell death following TSA treatment was significantly enhanced by Ad-E2F1 infection. In contrast, no such response was observed for cells infected with Ad-LacZ. These results are in agreement with the data from ER-E2F1 expressing cells and together with FIGS. 1b-c clearly indicate that E2F1 overexpression sensitizes HDACIs-induced cell death and that this cell death potentiation is independent of p53 status.

Selective Activation of E2F1 Pro-Apoptotic Targets by HDACIs.

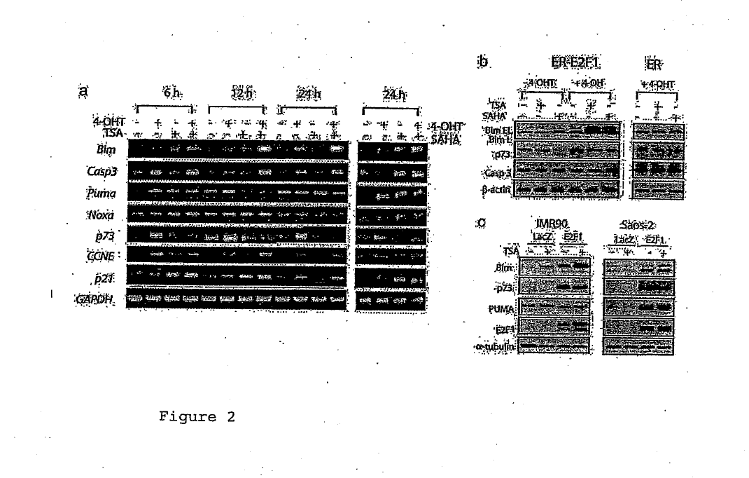

We reasoned that HDACIs might promote E2F1-mediated cell death, which prompted us to search for E2F1-regulated gene response to HDACIs. To achieve this aim, we used the microarray screening approach. BCL2L11, which encodes the pro-apoptotic BH3-only Bcl2 family member Bim (36, 37) and a recently identified E2F1 target (29), was found to be markedly induced by SAHA or TSA upon E2F1 activation (data not shown). To confirm the microarray data and to investigate whether other E2F1 pro-apoptotic targets are involved, we used RT-PCR to examine the expressions of a number of known E2F1 targets, including CCNE, p73, caspase-3, and the BH3-only proteins such as Puma, Noxa and Bim, in ER-E2F1 expressing cells treated with TSA or SAHA in the presence or absence of 4-OHT (FIG. 2a). Time-course analysis showed that addition of 4-OHT to ER-E2F1 expressing cells led to strong inductions of CCNE and p73 transcripts, ar to lesser extents, caspase-3, Bim, Puma and Noxa transcripts. Of these genes, 4-OHT-induced Bim and caspase-3 expressions were further upregulated by TSA or SAHA treatment (FIG. 2a). By contrast, expression of CCNE and other closely related BH3-only transcripts Puma and Noxa were, however, not notably upregulated. Intriguingly, the 4-OHT-induced p73 expression was paradoxically reduced by both TSA and SAHA. p21 activation, which is a typical response of HDACIs, was induced by TSA and SAHA as expected and this activation was not further increased in the presence of 4-OHT. These findings suggest that the HDAC inhibition alters the ability of E2F1 to activate transcription in a target gene-specific manner.

To determine whether these transcriptional changes will result in alterations in protein expression, we performed immunoblot analysis of Bim, Puma, caspase 3 and p73. FIG. 2b shows that treatment of ER-E2F1 increased Bim protein levels, compared to cells treated with TSA, SAHA or 4-OHT alone. Caspase-3 protein levels, as opposed to the striking increase in mRNA levels, were not notably increased by TSA and SAHA. Consistent with the mRNA analysis, Puma protein levels were not affected and the E2F1-dependent p73 induction was reduced by TSA and SAHA treatment. Thus, among the known E2F1 proapoptotic targets we have surveyed, Bim appears to be the primary E2F1 pro-apoptotic target whose expression is substantially upregulated by HDACIs in both mRNA and protein levels. This observation was further confirmed in normal human fibroblast IMR90 cells and osteosarcoma Saos-2 cells using Ad-E2F1. In both cases, TSA treatment of cells infected with Ad-E2F1 resulted in a marked increase in Bim expression compared with cells infected with Ad-LacZ (FIG. 2c). In contrast, p73 and Puma were not affected Collectively, these results suggest that HDACIs selectively activate E2F1 pro-apoptotic target Bim. Interestingly, this observation is in contrast to E2F1 activation by DNA damage, which results in selective induction of p73 (31, 38). Consistent with the known role of Bim in the mitochondria-mediated apoptosis, we observed increased activation of caspase-3 activity, increased PARP cleavage and the disruption of mitochondria membrane potential after SAHA or TSA treatment upon E2F1 activation (FIG. S1). Consistently, caspase 3 inhibitor partially blocked this apoptosis (FIG. S1).

Bim is Functionally Important in Conferring Sensitivity of HDACIs Upon E2F1 Activation.

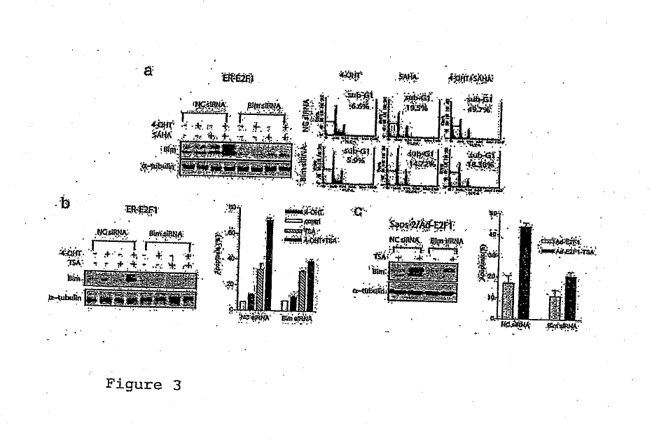

To definitively establish the role of Bim in HDACIs induced apoptosis upon E2F1 activation, we used RNA interference to silence Bim expression and analyzed its biological effects. To this end, we transfected ER-E2F1 expressing cells with Bim-specific siRNA and treated with SAHA for 24 h in the presence or absence of 4-OHT. As controls, cells were also transfected with an irrelevant siRNA. Western blot analysis of ER-E2F1 expressing cells showed that Bim-specific siRNA decreased E2F1-dependent Bim expression by more than 90%, and nearly completely abolished SAHA-induced further increase in Bim expression, as compared to the negative control siRNA (NC siRNA) treated cells (FIG. 3a, left panel). As a result, ER-E2F1 expressing cells treated with Bim siRNA showed a marked decrease in SAHA-induced apoptosis in the presence of 4-OHT (18%), as compared to the control siRNA treated cells (49%)(FIG. 3a, right panel). Thus, silencing of Bim induction by siRNA transfection resulted in effective abrogation of apoptosis enhancement by E2F1 in response to SAHA. Similar results were also obtained in TSA treated cells (FIG. 3b). Likewise, silencing of Bim expression also resulted in the decreased cell death response to TSA in Saos-2 cells infected with Ad-E2F1 (FIG. 3c). These concordant results show that Bim induction mediates the apoptosis potentiation upon E2F1 activation in response to HDACIs.

HDACIs Induce Bim Transcription Through Increased E2F1 Recruitment to the Bim Gene Promoter

BH3 only proteins including Bim have been proposed to be an E2F1 direct target. However, functional E2F1 binding sites in the human Bim promoter have not been previously identified. Sequence analysis of genomic sequence spanning 2.5 kb base pairs upstream of the transcription start site of the human Bim promoter revealed the presence of four sites similar to the consensus E2F binding motif [TTT(C/G)GCGC] at positions −1270/−1263, −1734/−1727, −2112/−2105, and −2245/−2238 from the transcription start site (FIG. 4a). To determine whether the human Bim promoter was responsive to E2F1, we isolated two genomic DNA fragments spanning the Bim promoter regions −1415/−205 and −2415/−1333 containing one and three putative E2F1 binding sites, respectively. The two fragments were cloned into the pGL3-luciferase reporter construct for analysis of promoter activity. The constructs were then cotransfected into HCT116 cells with an empty vector (pcDNA3.1), or increasing amount of E2F1 expressing plasmid together with a normalization control. Relative luciferase activity was measured after 48 h. The results indicate that E2F1 can induce up to 30-fold induction in promoter activity of the reporter construct that contains −1415/−205, suggesting that E2F1 is capable of activating the Bim −1415/−205 promoter. In contrast, Bim −2415/−1333 promoter had no response to E2F1 (FIG. 4b). Thus the putative E2F1-binding site (TTTGGCGG) within −1415/−205 appeared to be a functional E2F1 responsive element, denoted as E2F1-RE, in the Bim promoter.

To confirm that E2F1 binds to the E2F1-RE in vivo, we performed chromatin immunoprecipitation (CHIP) assays using an anti-E2F1 antibody and PCR primers encompassing the E2F1-RE (FIG. 4c, upper panel). The results show that E2F1 occupancy of the Bim E2F1-RE is readily detectable in ER-E2F1 expressing cells in the absence of 4-OHT, indicating endogenous binding of E2F1 to the Bim promoter. SAHA treatment promoted recruitment of endogenous E2F1 to the Bim promoter. In the presence of 4-OHT, E2F1 binding to the Bim promoter was significantly increased and this binding was further markedly increased by SAHA (FIG. 4c, lower panel). No signal above background was seen with nonspecific IgG. Thus, consistent with Bim being a direct target of E2F1, our study identified the functional E2F1 binding site in the human Bim promoter and further demonstrated that increases in E2F1 occupancy onto the Bim promoter after HDAC inhibition is associated with the upregulation of Bin expression.

Rb Inactivation Induces Bim Expression and Sensitization to HDACIs.

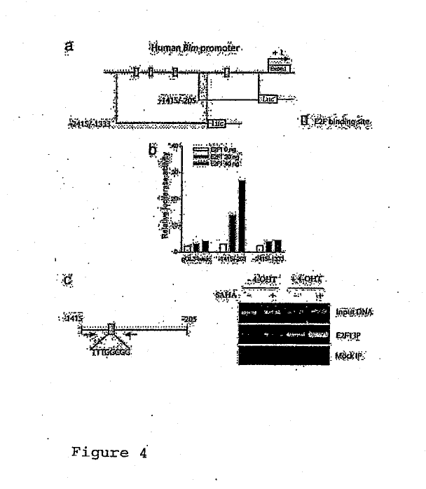

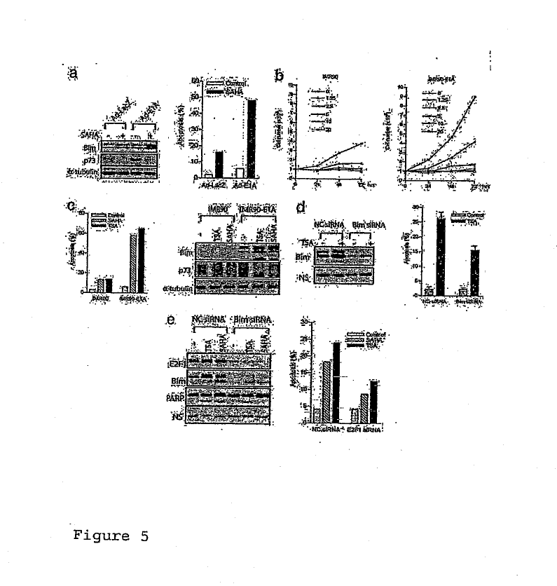

We next examined the apoptosis response to HDAC inhibition resulting from deregulation of endogenous E2F activity. E1A oncoprotein protein binds to and inactivates Rb family members (39, 40), resulting in the activation of the endogenous E2F1 (41, 42). To determine the effect of Rb inactivation on Bim expression and cellular response to HDACIs, we infected Rb wild-type U2OS cells with adenovirus expressing E1A or the control adenovirus. FIG. 5a shows that U2OS cells infected with Ad-E1A express higher Bim and p73, and after SAHA treatment, Bim but not p73 was further increased. Consistently, U2OS cells expressing E1A were much more sensitive to SAHA treatment compared with the U2OS cells infected with Ad-LacZ (FIG. 5a, right panel).

To determine the tumor selectivity of E2F1-Bim intervention, we compared the impacts of HDACIs on both proliferation and apoptosis in normal diploid human fibroblasts IMR90 cells and transformed IMR90 cells stably expressing E1A. A dose-response study showed that SAHA substantially inhibited proliferation of both normal and transformed IMR90 cells (FIG. 5b). However, normal IMR90 cells were basically not responsive to SAHA and TSA in inducing apoptosis (<10%), whereas the same treatments in E1A-transformed IMR90 cells triggered a strong apoptosis (˜60%) (FIG. 5c, left panel). This observation is consistent with the previous report using different cell lines (12) and suggest that the tumor selectivity of HDACIs occurs in the pathway(s) that regulate apoptosis rather than cell proliferation. Consistent with the higher E2F1 activity in IMR90-E1A cells, Bim and p73 were upregulated in these cells and after SAHA and TSA treatments Bim levels were further dramatically increased. In contrast, Bim induction was barely induced by TSA and SAHA in normal IMR90 cells (FIG. 5c, right panel). p73 expression, again, was reduced by SAHA; an observation consistent with E2F1-overexpressing cells. In addition, knockdown of Bim by siRNA in IMR90-E1A cells significantly reduced apoptosis induction in response to TSA and SAHA (FIG. 5d). Thus, oncogene EIA expression in normal IMR90 cells leading to the increased endogenous E2F1 activity sensitized these cells to SAHA-induced apoptosis at least in part through the induction of Bim.

We further used Rb-deficient Saos-2 cells to examine the definite role of endogenous E2F1 in apoptotic response and Bim induction following TSA or SAHA treatment. Saos-2 cells were transfected with the E2F1 specific siRNA or the negative control siRNA. FIG. 5e shows that E2F1 depletion in Saos-2 cells resulted in a substantial reduction of Bim expression after TSA or SAHA treatment and inhibited PARP cleavage. Consistently, SAHA and TSA-induced apoptosis was substantially reduced in E2F1 siRNA-treated cells (FIG. 5e, right panel). These results clearly demonstrate that endogenous E2F1 is required for the robust inductions of Bim and apoptosis in response to HDACIs.

Transcriptional Apoptotic Network Regulated by SAHA and E2F1.

In the previous study, we have reported that HDACIs activates E2F1-dependent apoptosis and that E2F1 target Bim plays a crucial role min this process. To fully characterize the genomic program and to identify additional molecular events that might participate in E2F1-dependent apoptosis in response to HDAC inhibition, we employed DNA microarray analysis and an E2F1-inducible HCT116 p53 null cell line (63). In this cell system, E2F1 is fused to the estrogen receptor (ER)-binding domain (64) and ER-E2F1 is activated by ER-ligand 4-hydroxytamoxifen (4-OHT). As described previously (63) and as shown here in FIG. 1A, HDAC inhibitor SAHA induced markedly increased apoptosis upon E2F1 activation by 4-OHT.

To determine the apoptotic program that contributes to the SAHA-induced apoptosis as a result of E2F1 activation, we focused on 220 well-annotated apoptosis-related genes represented in the 19k gene array. We began by defining apoptotic genes activated by 4-OHT over a 4, 8 and 24 h time course in ER-E2F1 expressing cells or cells expressing the empty vector that contain ER-binding domain only (ER). Gene expression data from each time points was cluster analyzed and displayed in the Tree view software (62). As shown in FIG. 6B, 42 of 220 apoptosis-related genes showed increased expression after 4-OHT treatment in ER-E2F1 cells, but not in ER cells, indicating these apoptotic genes are potentially regulated by E2F1. Among E2F1 regulated apoptotic genes, BCl2L11, TP73, CASP3 are previously known E2F1 targets and were strongly upregulated by E2F1. In addition, we found for the first time that RUNX3, ATM, TP53BPL, RPS6KA1 were also substantially activated by E2F1.

To identify SAHA responsive genes that reflect the E2F1 apoptotic activity, we next compared the expression patterns of apoptosis genes commonly and selectively activated by SAHA treatment in the presence and absence of 4-OHT. 49 genes were found to be upregulated by SAHA for at least one time point regardless of the presence of 4-OHT (FIG. 6C). As previously described, we found that SAHA strongly activated multiple genes implicated in apoptosis program. Among them are those involved in intrinsic apoptotic pathway (65), such as Bcl2-family members (BCl2L11, BNIP3L, BCL10), APAF1 and CASP3. In addition, genes belonging to the receptor-mediated death pathway (TNFRSF1B, TNFRSF10D and TNFSF13) were also induced by SAHA, albeit to lesser extents. Among them, 15 were E2F1 targets as marked in FIG. 6C. We reasoned that if SAHA activates E2F1 apoptotic program, then we should find a set of genes whose expressions can be further induced by SAHA upon E2F1 activation. Indeed, cluster analysis revealed a subset of genes whose expressions were markedly enhanced by SAHA upon E2F1 activation by 4-OHT (FIG. 6C, cluster A). Notably, in cluster A of FIG. 6C, MAP3K5, which encodes apopotosis-stimulating kinase 1(ASK1) and appeared to be weakly induced by E2-F1 alone, was strongly unregulated by SAHA upon E2F1 activation. In the same cluster were BCl2L11 (Bim) and CASP3 that had been described in our previous study (63). These observations support two conclusions: (1) SAHA activates E2F1 apoptotic activity through a target gene-specific manner. (2) ASK1 is E2F1-regulated target and its expression can be activated by SAHA.

Both Exogenous and Endogenous E2F1 Regulates ASK1 expression.

To confirm the results obtained from the gene expression analysis, RT-PCR experiments were performed to examine the ASK1 mRNA levels in HCT116 cells expressing ER-E2F1, ER or a DNA-binding-defective mutant of E2F1 (E132) fused to ER. In agreement with the microarray data, activation of E2F1 by 4-OHT resulted in an increase in ASK1 mRNA level, but not in cells expressing ER or ER-E132 (FIG. 7A, left panel). Consistently, ASK1 protein was accumulated following 4-OHT in ER-E2F1 expressing cells (FIG. 7A, right panel). To investigate whether ASK1 expression is associated with the endogenous E2F1, we arrested the human osterosarcoma cells U2OS in G0/G1 phase by serum starvation (FIG. 7B, left panel). Expression of E2F1 was elevated when serum-starved U2OS cells reentered the cell cycle following serum addition (FIG. 7B, right panel). Corresponding to the enhanced E2F1 activity, we detected an increase in ASK1 expression as cells entered S phase, together with the bona-fide E2F1 targets p73 and Cyclin E. Thus, ASK1 expression is correlated with the endogenous E2F1 expression. To further substantiate this conclusion, we also used the adenovirus expressing oncoprotein EIA (Ad-E1A) to infect U2OS and IMR90 cells. E1A binds to and inactivates Rb family members (59, 66), resulting in the activation of the endogenous E2F1 (67, 68). Thus, cells overexpressing E1A will have enhanced E2F1 activity and thus increased expression of E2F1 target genes. Indeed, we found that Ad-E1A infection resulted in the upregulation of ASK1 and p73 in both U2OS and IMR90 cells (FIG. 7C). These observations suggest that activation of endogenous E2F1 is able to induce the expression of ASK1.

ASK1 is a Direct Target of E2F1.

To determine whether the E2F1 induction of ASK1 is regulated at the level of transcription, the ASK1 promoter was isolated and subcloned into a luciferase reporter plasmid. FIG. 8A illustrates the promoter region of the ASK1 gene, including the putative E2F binding sites as well as the deletion mutant for reporter constructs. As can be seen in FIG. 8B, the promoter activity of the 1.0 kb of 5′-proximal region of ASK1 gene can be markedly activated by increasing amounts of E2F1 plasmid. To determine the potential E2F-binding region that mediates the induction, we next measured ASK1 promoter activity using various deletion mutants. The deletion constructs containing region between −1000 and −256 was not responsive to E2F1 (FIG. 8C). In contrast, E2F1 can activate the −273/+125 promoter for up to 12-fold, suggesting that the putative E2F1−binding site within −273/+125 is a functional E2F1 responsive element in ASK1 promoter.

To examine the in vivo recruitment of E2F1 to the ASK1 gene promoter, we used the ER-E2F1 or a DNA-binding deficient mutant (ER-E132) expressing cells to perform the chromatin immunoprecipitation (ChIP) assay. In the absence of 4-OHT, anti-E2F1 immunoprecipitated the proximal region of ASK1 gene promoter from −273 to +125, containing the E2F1 motif in both ER-E2F1 or ER-E132 expressing cells, whereas control IgG did not (FIG. 8D). This indicates that endogenous E2F1 binds to the ASK1 promoter. In the presence of 4-OHT, which activates ER-E2F1, recruitment of E2F1 to the ASK1 promoter was substantially increased in ER-E2F1 cells, whereas the activation of binding mutant ER-E132 failed to do so. In light of these results, we conclude that E2F1 activates ASK1 transcription and ASK1 is a direct target of E2F1.

ASK1 Regulates E2F1 Activity Through a Positive Feedback Mechanism

E2F1 activity is negatively regulated by pRb. pRb hyperphosphorylation inactivates Rb, resulting in increased E2F1 activity. ASK1 has been recently shown to physically interact and inactivate pRB (69). This observation raises the possibility that ASK1 induction by E2F1 might lead to pRB inhibition and thus provide a positive feedback loop on E2F1 activity. Given this possibility, we determined whether ASK1 knockdown by siRNA could impair the activation of E2F1 targets. In ER-E2F1 expressing cells, activation of E2F1 by 4-OHT resulted in the induction of ASK1 as well as other E2F1 targets p73, Bim and Cyclin E. We found that depletion of ASK1 by siRNA not only prevented its own induction by 4-OHT but also substantially impaired the induction of p73, Bim and cyclin E (FIG. 9A). This result suggests that E2F1-mediated ASK1 induction permits a positive feedback effect on E2F1 activity, leading to the sufficient induction of E2F1 target genes.

To investigate whether ASKS activates E2F1 activity through inhibition of Rb, we used the E2F1-responsive Bim promoter to test the effect of ASK1 on E2F1/Rb-mediated regulation on Bim promoter activity. In the luciferase reporter assay, ecotopic expression of E2F1 activated Bim promoter and as expected this E2F1 function was inhibited by co-expression of pRB (FIG. 9B). Further introduction of ASK1 expression plasmid reversed the negative effect of Rb on E2F1-mediated activation of Bim promoter (FIG. 9B). Thus, these results support the conclusion that ASK1 induction by E2F1 results in Rb inactivation, and thereby increases E2F1 activity through a positive feedback loop.

SAHA Promotes E2F1-Mediate ASK1 Induction.

Microarray analysis as shown in FIG. 6 indicates that ASK1, like Bim, is weakly regulated by E2F1 and this regulation, however, can be significantly augmented following HDAC inhibition by SAHA. This observation is further validated through Western blot analysis in ER-E2F1 expressing cells (FIG. 10A). To examine whether the increased ASK1 induction by SAHA upon E2F1 activation is associated with the increased E2F1 recruitment to the ASK1 promoter, we performed the ChIP assay. Indeed, under the SAHA treatment in the presence of 4-OHT, E2F1 binding to the ASK1 promoter was markedly increased (FIG. 10B).

To investigate whether the endogenous E2F1 is required for ASK1 induction by SAHA, we compared the IMR90 and IMR90 cells stably expressing E1A oncoprotein. Consistent with the higher E2F1 activity in IMR90-E1A cells, these cells express higher level of ASK1 as compared to the IMR90 cells and after SAHA treatment ASK1 expression was further induced (FIG. 10C). To determine the definite role of E2F1 in SAHA-induced ASK1 expression, we used the E2F1 siRNA to inhibit E2F1 expression in U2OS cells and examined its effect on ASK1 expression after SAHA treatment. FIG. 10D shows that SAHA induced ASK1 expression over time in U2OS cells and cells treated with E2F1 siRNA but not control siRNA efficiently abolished ASK1 induction. Taken together, these results indicate that both exogenous and endogenous E2F1 is required for the induction of ASK1 in response to SAHA.

E2F1-ASK1 Activation Contributes to Apoptosis Induction by SAHA

We next examined the role of ASK1 in SAHA-induced apoptosis resulting from enhanced E2F1 activity. To this end, we used ASK1 siRNA to reduce the ASK1 expression in ER-E2F1 expressing cells and observed a marked reduction in the level of apoptosis following SAHA treatment upon E2F1 activation (FIG. 11A). Consistent with the role of ASK1 on the positive feedback regulation on E2F1 activity, we found that ASK1 depletion resulted in marked reduction of Bim in response to SAHA (FIG. 11B). We have previously demonstrated that E2F1-Bim pathway plays an important role in HDACI-induced cell death. Thus, inhibition of ASK1 expression by siRNA can reduce SAHA apoptotic response through impairing the induction of Bim. This observation suggests that the active engagement of the ASK1-E2F1 feedback module is necessary to promote the apoptosis in response to HDACIs at least in part through the increased E2F1-Bim activity.

The ASK1 protein connects to several differs intracellular signal transduction pathways including JNK and the p38 mitogen-activated protein kinase (MAPK) family leading to apoptosis in embryonic fibroblast and pheochromocytoma cells (70, 71). Given that ecotopic E2F1 expression results in ASK1 activation and promotes SAHA-induced apoptosis, we examined the JNK-p38 activation following E2F1 activation and SAHA treatment. The activation of JNK and p38 MAPK in response to SAHA was monitored by immunoblotting using phospho-specific antibody to detect JNK or p38 activation (FIG. 11B). In ER-E2F1 expressing cells, we did not observe a consistent increase in levels of phosphor-p38 or phosphor-JNK following E2F1 activation, SAHA treatment or both, indicating the increased apoptosis is not due to the increased JNK or p38 activation.

Discussion

HDAC inhibitors are considered to be promising chemotherapeutic agents due to their selective activity toward cancer cells. However, the basis for tumor selectivity of these compounds is one of the unsolved questions (2). The results described here establish the oncogenic Rb/E2F1 pathway as a target for HDACIs and demonstrate that HDACIs triggers efficient apoptosis through activation of E2F1 apoptotic function. Importantly, we identified Bim as a critical mediator in this process. That Rb/E2F pathway is frequently deregulated in many types of cancers and that HDACIs preferentially kill tumor cells carrying enhanced E2F1 activity, define a set of molecular conditions for selectivity of anti-tumor effects of HDACIs. Taken together, we posit that tumors with defective pRb and confirmed upregulation of the E2F1 pathway would be specifically sensitive to HDAC inhibitors.

Like that of oncoprotein Myc, E2F1 functions not only as an oncogene to stimulate cell cycle progression and elicit proliferation, but is also equipped with a tumor suppressor function by inducing apoptosis. This failsafe mechanism protects aberrant oncogenic transformation of normal cells, and in cancer cells this mechanism is tightly controlled or disabled to allow malignant outgrowth. Therefore, therapeutic approaches for fully restoration or activation of oncogene-induced apoptosis appear to be conceptually feasible to achieve tumor-specific intervention. Thus, elicitation of E2F1-mediated tumor suppressor function through HDAC inhibition without causing DNA damage may be an attractive strategy to achieve cancer specific killing. The therapeutic benefit by utilizing this strategy is obvious: it selectively kills tumor cells and spares the normal tissue.

We identified Bim as a key mediator of E2F1-induced apoptosis provoked by HDACIs. Among previously identified E2F1 proapoptotic targets that have been served using both microarray and RT-PCR, Bim was found to be dramatically increased in both mRNA and protein levels by HDACIs upon E2F1 activation. Moreover, silencing of Bim expression by RNAi efficiently abrogated the cell death enhancement induced by HDACIs in E2F1-overexpressing cells, indicating that the sole up-regulation of Bim is sufficient to drive these cells into strong apoptosis. Bim is a proapoptotic BH3 domain-only member of the Bcl-2 family and can trigger intrinsic apoptosis pathway through activation of Bax (36, 43, 44). Consistently, HDACIs treatment results in a marked increase in the caspase 3 activity and PARP cleavage as well as the disruption of mitochondrial membrane potential in E2F1-overexpressing cells. However, we do not exclude the possibility that induction of other previously unidentified E2F1 targets might also contribute to the sensitization of apoptosis. Nevertheless, the nearly complete abrogation of apoptosis sensitization by Bim siRNA indeed indicates that Bim plays a central role in this process.

An intriguing observation is that the other BH3-only proteins such as Puma and Noxa are not affected by HDAC inhibition, albeit they are also previously identified E2F1 targets (29). The molecular basis that dictates the selective activation of E2F1 target genes by HDACIs is not known. It has been previously reported that E2F1 activity can be modulated through increased acetylation at lysine residues at 117, 120, and 125 through histone acetyltransferase (HAT) activity of PCAF (45, 46). Thus, the selective E2F1 target activation might be associated with increased acetylation of E2F1 since the acetylation of transcription factors such as p53 and p73 were known to lead to the selective activation of proapoptotic targets (47, 48). However, we found that acetylation-deficient mutant ER-E2F1 carrying an alteration of the three lysines to arginine did not interfere with its ability to transactivate Bim as well as the apoptotic response to HDACI (data not shown). Thus, such an effect of HDACIs is not likely the result of E2F1 acetylation itself. In principle, it could be due to either the acetylation of proteins physically associated with E2F1 or increased recruitment of HATs and subsequent acetylating of histones of affected promoters.

Previously, studies show that DNA damage promotes E2F1-mediated apoptosis through selective induction of p73 (31, 38) or activation of Chk2-p53 network (25). In striking contrast to that induced by DNA d we demonstrate that activation of E2F1 apoptotic function by HDACI does not require either p73 or p53, but proceeds through robust activation of pro-apoptotic Bcl-2 family member Bim. Unlike other BH-3 only members such as Puma, Noxa and Bid that are p53 targets and participate in DNA damage-induced apoptosis (49-52), there is no evidence that Bim is a p53 target Instead, its expression is tightly regulated by growth factor signals through phosphatidylinositol 3-OH kinase (P3K)/protein kinase B (PKB)/Akt (53, 54) or ERK/mitogen-activated protein kinase (MAPK) pathway (55-57) and participate in apoptosis induced by cytokine withdraw. Thus, Bim appears to be separated out from other members of BH-3 only proteins and emerged as a key mediator of oncogene-induced apoptosis, whereas Puma, Noxa, and Bid are primarily involved in DNA damage and p53-mediated apoptotic response. Indeed, it was recently reported that loss of Bim facilitates oncogene Myc-induced tumorigenesis (58), though it is not clear whether Bim is a direct target of Myc. Taken together, Bim appears to be a key mediator of oncogene-induced apoptosis. It is thus expected that in tumors where p53 is lost, Bim-mediated apoptotic pathway might be key to couple oncogenic lesions in order to achieve the fail-safe homeostatic mechanism. In addition, our results indicate that HDACIs are not likely subject to chemoresistance from p53 mutations cancer cells.

In summary, we have evidence to show that increased expression of E2F1 or Rb inactivation results in a strong potentiation of HDACI-induced apoptosis. Importantly, this has been demonstrated in normal versus transformed cells and strongly supports a potential mechanism for tumor selectivity of HDACIs. In addition, Bim might be a more valid predictive marker that can determine HDACI response, as opposed to the currently used acetylated histones, since HDACIs induce hyperacetylation of histones in both tumor and normal tissues. Using HDACIs to promote E2F1-mediated but p53-independent apoptosis provides the proof of concept that restoration of oncogene-induced apoptosis without causing DNA damage is a feasible strategy for cancer specific therapy.

To identify additional gene elements that might be involved in this process, we interrogated the genomic response of HDACI inhibitor SAHA and focused on the gene expression changes of 220 apoptosis-related genes. Our study is in line with the previous studies showing that HDACIs affect the expression of multiple genes involved in both the intrinsic apoptotic and receptor-mediated apoptotic pathways (65). Consistent with a possible role of E2F1 in SAHA response, a number of E2F1 targets, such as BCl2L11 (encodes Bim), CASP3, APAF1, TNFRSF10D, MAP3K5 (encodes ASK1) that have been identified either by previous studies or this study, are activated by SAHA. Importantly, upon E2F1 activation by 4-OHT, inductions of Bim, caspase 3 and ASK1 are further increased by SAHA. Thus, in addition to the previously identified Bim and caspase 3, ASK1 appears to be another target gene whose product might participate in the enhanced HDACI response resulting from E2F1 activation.

We show here, using RNA interference as well as a dominant-negative mutant of E2F, that ASK1 is a direct target of E2F1 and E2F1 activity is required for the ASK1 induction by SAHA. ASK1 is involved in multiple signaling pathways leading to apoptosis (70, 71). It has been reported that ASK1-mediated apoptosis is mediated through the phosphorylation and activation of proapoptotic p38 and JNK signaling pathway in some cellular system. In fact, we did not observe an increased p38 or JNK phosphorylation in response to SAHA upon E2F1 activation, suggesting that the conventional role of ASK1 in apoptotic function is not contributing to this process. Importantly, we show that ASK1 knockdown by RNA interference impaired the induction of other E2F1 targets including Bim, p73 and cyclin E. We thus propose that an important function of ASK1 induction lies in the feedback regulation of E2F1 activity.

E2F1 activity is regulated via various upstream components, including Rb, p16 and cdk activity. In addition, E2F1 activity might also be regulated through a feedback mechanism mediated through its target genes. For instance, it has been shown that E2F1 induces the expression of cdk inhibitor p27, resulting in a negative feedback regulation on E2F1 transcriptional activity through inhibition of cdk activity and Rb hyperphosphorylation (72). Consistent with a previous-report that ASK1 is inhibitory for Rb function (69), we show that Rb-mediated repression of Bim promoter activation by E2F1 can be reversed by ASK1 overexpression. We propose that ASK1 induction by E2F1-provides a positive feedback regulation on E2F1 through Rb inhibition. Thus, E2F1 can be regulated by both positive and negative-feedback mechanism through its target genes. The suppression of Bim induction and inhibition of apoptosis induction by SAHA following ASK1 knockdown suggests that ASK1 induction contributes to SAHA-induced cell death by potentiating E2F1-Bim apoptotic network. Although the role of ASK1 in E2F1-mediated apoptosis need to be further evaluated, our data suggest that the concomitant inductions of ASK1 and Bim reflect the efficiency of the mechanism through which E2F supports the sustained Bim induction that is central to inducing apoptosis.

It will be understood that the invention has been described by way of example only and modifications may be made whilst remaining within the scope and spirit of the invention.

### REFERENCES

1. Gabrielli, B. G., Johnstone, R. W. & Saunders, N. A. (2002) Curr Cancer Drug Targets 2, 337-53.
2. Johnstone, R. W. & Licht, J. D. (2003) Cancer Cell 4, 13-8.
3. Marks, P. A., Richon, V. M., Breslow, R. & Rifkind, R. A. (2001) Curr Opin Oncol 13, 477-83.
4. Melnick, A. & Licht, J. D. (2002) Curr Opin Hematol 9, 322-32.
5. He, L. Z., Tolentino, T., Grayson, P., Zhong, S., Warrell, R. P., Jr., Rifkind, R. A., Marks, P. A., Richon, V. M. & Pandolfi, P. P. (2001) J Clin Invest 108, 1321-30.
6. Warrell, R. P., Jr. (1999) Haematologica 84 Suppl EHA-4, 75-7.
7. Kelly, W. K., O'Connor, O. A., Krug, L. M., Chiao, J. H., Heaney, M., Curley, T., MacGregore-Cortelli, B., Tong, W., Secrist, J. P., Schwartz, L., Richardson, S., Chu, E., Olgac, S., Marks, P. A., Scher, H. & Richon, V. M. (2005) J Clin Oncol 23, 3923-31.
8. Yoshida, M., Kijima, A, Akita, M.& Beppu, T. (1990) J Biol Chem 265, 17174-9.
9. Richon, V. M., Emiliani, S., Verdin, E., Webb, Y., Breslow, R. Rifkind, R. A. & Marks, P. A. (1998) Proc Natl Acad Sci USA 95, 3003-7.
10. Nebbioso, A., Clarke, N., Voltz, E., Germain, E., Ambrosino, C., Bontempo, P., Alvarez, R, Schiavone, E. M., Ferrara, F., Bresciani, F., Weisz, A., de Lera, A. R., Gronemeyer, H. & Altucci, L. (2005) Nat Med 11, 77-84.
11. Insinga, A., Monestiroli, S., Ronzoni, S., Gelmetti, V., Marchesi, F., Viale, A., Altucci, L., Nervi, C., Minucci, S. & Pelicci P. G. (2005) Nat Med 11, 71-6.
12. Ungerstedt, J. S., Sowa, Y., Xu, W. S., Shao, Y., Dokmanovic, M., Perez, G., Ngo, L., Holmgren, A., Jiang, X. & Marks, P. A. (2005) Proc Natl Acad Sci USA 102, 673-8.
13. Roy, S., Packman, K., Jeffrey, R. & Tenniswood, M. (2005) Cell Death Differ.
14. Lowe, S. W., Cepero, E. & Evan, G. (2004) Nature 432, 307-15.
15. Sherr, C. J. & McCormick, F. (2002) Cancer Cell 2, 103-12.
16. Sherr, C. J. (1996) Science 274, 1672-7.
17. Zhang, S. Y., Liu, S. C., Al-Saleem, L. F., Holloran, D., Babb, J., Guo, X. & Klein-Szanto, A. J. (2000) Cancer Epidemiol Biomarkers Prev 9, 395-401.
18. Gorgoulis, V. G., Zacharatos, P., Mariatos, G., Kotsinas, A., Bouda, M., Kletsas, D., Asimacopoulos, P. J., Agnantis, N., Kittas, C. & Papavassiliou, A. G. (2002) J Pathol 198, 142-56.
19. Etges, A., Nunes, F. D., Ribeiro, K C. & Araujo, V. C. (2004) Oral Oncol 40, 326-31.
20. Cam, H. & Dynlacht, B. D. (2003) Cancer Cell 3, 311-6.
21. Ginsberg, D. (2002) FEBS Lett 529, 122-5.
22. Matsumura, I., Tanaka, H. & Kanakura, Y. (2003) Cell Cycle 2, 333-8.
23. Zhu, J. W., DeRyckere, D., Li, F. XY, Wan, Y. Y. & DeGregori, J. (1999) Cell Growth Differ 10, 829-38.
24. Bates, S., Phillips, A C., Clark, P. A., Stott, F., Peters, G., Ludwig, R. L. & Vousden, K. H. (1998) Nature 395, 124-5.
25. Rogoff, H. A, Pickering, M. T., Frame, F. M., Debatis, M. E., Sanchez, Y., Jones, S. & Kowalik, T. F. (2004) Mol Cell Biol 24, 2968-77.
26. Stiewe, T. & Putzer, B. M. (2000) Nat Genet 26, 464-9.
27. Irwin, M., Marin, M C., Phillips, A. C., Seelan, R. S., Smith, D. I., Liu, W., Flores, E. R, Tsai, K Y, Jacks, T., Vousden, K. H. & Kaelin, W. G., Jr. (2000) Nature 407, 645-8.
28. Nahle, Z., Polakoff, Davuluri, R. V. McCurrach M. E., Jacobson, M. D., Narita, M., Zhang, M. Q., Lazebnik, Y., Bar-Sagi, D. & Lowe, S. W. (2002) Nat Cell Biol 4, 859-64.
29. Hershko, T. & Ginsberg, D. (2004) J Biol Chem 279, 8627-34.
30. Vigo, E., Muller, H., Prosperini, E., Hateboer, G., Cartwright, P., Moroni, M. C. & Helin, K (1999) Mol Cell Biol 19, 6379-95.
31. Urist, M., Tanaka, T., Poyurovsky, M. V. & Prives, C. (2004) Genes Dev 18, 3041-54.
32. Inoue, K., Roussel, M. F. & Sherr, C. J. (1999) Proc Natl Acad Sci USA 96, 3993-8.
33. Robertson, K D. & Jones, P. A. (1998) Mol Cell Biol 18, 6457-73.
34. Stanelle, J., Stiewe, T., Theseling, C. C., Peter, M. & Putzer, B. M. (2002) Nucleic Acids Res 30, 1859-67.
35. Ohtani, K., DeGregori, J. & Nevins, J. R (1995) Proc Natl Acad Sci USA 92, 12146-50.
36. Bouillet, P., Purton, J. F., Godfrey, D. I., Zhang, L. C., Coultas, L., Puthalakath, H., Pellegrini, M., Cory, S, Adams, J. M. & Strasser, A. (2002) Nature 415, 922-6.
37. O'Connor, L., Strasser, A., O'Reilly, L. A, Hausmann, G., Adams, J. M., Cory, S. & Huang, D. C. (1998) Embo J 17, 384-95.
38. Pediconi, N, lanai, A., Costmazo, A., Belloni, L., Gallo, R., Cimino, L., Porcellini, A., Screpanti, I., Balsano, C., Alesse, E., Gulino, A. & Levrero, M. (2003) Nat Cell Biol 5, 552-8.
39. Sherr, C. J. (2001) Nat Rev Mol Cell Biol 2, 731-7.
40. Dyson, N. (1998) Genes Dev 12, 2245-62.
41. Macleod, K. F., Hu, Y. & Jacks, T. (1996) Embo J 15, 6178-88.
42. Hurford, R K., Jr., Cobrinik, D., Lee, M. H. & Dyson, N. (1997) Genes Dev 11, 1447-63.
43. Gilley, J., Coffer, P. J. & Ham, J. (2003) J Cell Biol 162, 613-22.
44. Essafi, A., Fernandez de Mattos, S., Hassen, Y. A., Soeiro, I., Mufti, G. J., Thomas, N. S., Medema, R. H. & Lam, E. W. (2005) Oncogene.
45. Martinez-Balbas, M. A., Bauer, U. M., Nielsen, S. J., Brehm, A. & Kouzarides, T. (2000) Embo J 19, 662-71.
46. Marzio, G., Wagener, C., Gutierrez, M. I., Cartwright, P., Helin, K. & Giacca, M. (2000) J Biol Chem 275, 10887-92.
47. Costanzo, A., Merlo, P., Pediconi, N., Fulco, M., Sartorelli, V., Cole, P. A., Fontemaggi, G., Fanciulli, M., Schiltz, L., Blandino, G., Balsano, C. & Levrero, M. (2002) Mol Cell 9, 175-86.
48. Terui, T., Murakami, K., Takimoto, R., Takahashi, M., Takada, K., Murakami, T., Minami, S., Matsunaga, T., Takayama, T., Kato, J. & Niitsu, Y. (2003) Cancer Res 63, 8948-54.
49. Villunger, A., Michalak, E. M., Coultas, L., Mullauer, F., Bock, G., Ausserlechner, M. J., Adams, J. M. & Strasser, A. (2003) Science 302, 1036-8.
50. Nakano, K. & Vousden, K. H. (2001) Mol Cell 7, 683-94.
51. Yu, J., Wang, Z., Kinzler, K. W., Vogelstein, B. & Zhang, L. (2003) Proc Natl Acad Sci USA 100, 19311-6.
52. Sax, J. K., Fei, P., Murphy, M. E., Bernhard, E., Korsmeyer, S. J. & El-Deiry, W. S. (2002) Nat Cell Biol 4, 842-9.
53. Dijkers, P. F., Birkenkamp, K. U., Lam, E. W., Thomas, N. S., Lammers, J. W., Koenderman, L. & Coffer, P. J. (2002) J Cell Biol 156, 531-42.
54. Dijkers, P. F., Medema, R H., Lammers, J. W., Koenderman, L. & Coffer, P. J. (2000) Curr Biol 10, 1201-4
55. Reginato, M. J., Mills, K. R, Paulus, J. K., Lynch, D. K, Sgroi, D. C., Debnath, J., Muthuswamy, S. K. & Brugge, J. S. (2003) Nat Cell Biol 5, 733-40.
56. Shinjyo, T., Kuribara, R, Inukai T., Hosoi, H., Kinoshita, T., Miyajima, A., Houghton, P. J., Look, A. T., Ozawa, K. & Inaba, T. (2001) Mol Cell Biol 21, 854-64.
57. Weston, C. R, Balmanno, K., Chalmers, C., Hadfield, K., Molton, S. A., Ley, R., Wagner, E. F. & Cook, S. J. (2003) Oncogene 22, 1281-93.
58. Egle, A, Harris, A. W., Bouillet, P. & Cory, S. (2004) Proc Natl Acad Sci USA 101, 6164-9.
59. Dyson, N. (1998) Genes Dev 12, 2245-2262
60. Ishida, S., Huang, E., Zuzan, H., Spang, R., Leone, G., West, M., and Nevins, J. R (2001) Mol Cell Biol 21, 4684-4699
61. Yu, Q., He, M., Lee, N. H., and Liu, E. T. (2002) J Biol Chem 277, 13059-13066
62. Eisen, M. B., Spellman, P. T., Brown, P. O., and Botstein, D. (1998) Proc Natl Acad Sci USA 95, 14863-14868
63. Zhao, Y., Tan, J., Zhuang, L., Jiang, X., Liu, E. T., and Yu, Q. (2005) Proc Natl Acad Sci USA
64. Vigo, E., Muller, H., Prosperini, E., Hateboer, G., Cartwright, P., Moroni, M. C., and Helin, K. (1999) Mol Cell Biol 19, 6379-6395
65. Peart, M. J., Smyth, G. K, van Laar, R K, Bowtell, D. D., Richon, V. M., Marks, P. A., Holloway, A. J., and Johnstone, R. W. (2005) Proc Natl Acad Sci USA 12, 3697-3702
66. Sherr, C. J. (2001) Nat Rev Mol Cell Biol 2, 731-73-7
67. Macleod, K. F., Hu, Y., and Jacks, T. (1996) Embo J 15, 6178-6188
60. Hurford, R. K., Jr., Cobrinik, D., Lee, M. H., and Dyson, N. (1997) Genes Dev 11, 1447-1463
69. Dasgupta, P., Betts, V., Rastogi, S, Joshi, B., Morris, M., Brennan, B., Ordonez-Ercan, D., and Chellappan, S. (2004) J Biol Chem 279, 38762-38769
70. Tobiume, K., Matsuzawa, A, Takahashi, T., Nishitoh, H., Morita, K., Takeda, K, Minowa, O., Miyazono, K., Noda, T., and Ichijo, H. (2001) EMBO Rep 2, 222-228
71. Mizumura, K, Takeda, K., Hashimoto, S., Horie, T., and Ichijo, H. (2005) J Cell Physiol
72. Wang, C., Hou, X., Mohapatra, S., Ma, Y., Cress, W. D., Pledger, W. J., and Chen, J. (2005) J Biol Chem 280, 12339-12343

1. A method for assessing the suitability of a cancer patient for treatment with a histone deacetylase inhibitor, the method comprising assaying a biological sample from the patient for elevated E2F1 activity.

2. A method for selecting a cancer patient for treatment with a HDACI, the method comprising selecting a patient who has assayed positive for elevated E2F1 activity.

3. A method for treating a cancer patient wherein the method comprises treating the patient with a HDACI and wherein the patient's cancer has assayed positive for elevated E2F1 activity.

4. A method according to any of the preceding claims wherein the cancer may be selected from the group consisting of: retinoblastoma, breast cancer, lung cancer (e.g. non-small lung cancer or small cell lung cancer), salivary gland tumor, pancreatic cancer, glioblastoma multiforma and mantle cell lymphoma.

5. A method according to any of the preceding claims wherein the one or more biological sample includes tissues, cells, body fluids and isolates thereof etc., isolated from the cancer patient, as well as tissues, cells and fluids etc., present within a subject.

6. Use of a HDACI in the manufacture of a medicament for the treatment of a cancer patient whose cancer has assayed positive for elevated E2F1 activity.

7. A kit for use in a method of any of the preceding claims, wherein the kit comprises one or more reagents for use in assessing E2F1 activity in a biological sample.

8. A kit according to claim 7, wherein the kit comprises one or more components selected from the group consisting of:
(a) a labelled compound or agent capable of detecting a marker protein or nucleic acid in a sample;
(b) means for determining the level of the marker protein or marker nucleic acid in the sample (e.g., an antibody which binds the protein or a fragment thereof, or an oligonucleotide probe which binds to DNA or mRNA encoding the protein);
(c) instructions for interpreting the results obtained using the kit;
(d) a buffering agent;
(e) a preservative;
(f) a protein stabilizing agent;
(g) components for use in detecting the detectable label (e.g., an enzyme or a substrate); and
(h) software, for example software for selecting patient treatment.
